SEQUENCE LISTING (12 sequences)

SEQ ID NO: 1 - protein, 371 aa, Vaccinia virus
MDKLRVLYDEFVTISKDNLERETGLSASDVDMDFDLNIFMTLVPVLEKKVCAITPTIEDDKIVTMMKYCSYQSFSFWFLKSGAVVKSVYNKLDYVKKEKFVATFRDMLLNVQTLISLNSMYTRLRQDTEDIVSDSKKIMEIVSHLRASTTENAAYQVLQQNNSFIISTLNKILSDENYLLKIIAVFDSKLISEKETLNEYKQLYTISSESLVYGIRCSNLDISSVQLSNNKYVLFVKKMLPKIILFQNNDINAQQFANVISKIYTLIYRQLTSNVDVGCLLTDTIESAKTKISVEKIKQTGINNVQSLIKFISDNKKKYKTIISEEYLSKEDRIITILQDIVNEHDIKYDNKLLNMRDLIVTFRERYSYKF

SEQ ID NO: 2 - protein, 637 aa, Vaccinia virus
MNTGIIDLFDNHVDSIPTILPHQLATLDYLVRTIIDENRSVLLFHIMGSGKTIIALLFALVASRFKKVYILVPNINILKIFNYNMGVAMNLFNDEFIAENIFIHSTTSFYSLNYNDNVINYNGLSRYNNSIFIVDEAHNIFGNNTGELMTVIKNKNKIPFLLLSGSPITNTPNTLGHIIDLMSEETIDFGEIISRGKKVIQTLLNERGVNVLKDLLKGRISYYEMPDKDLPTIRYHGRKFLDTRVVYCHMSKLQERDYMITRRQLCYHEMFDKNMYNVSMAVLGQLNLMNNLDTLFQEQDKELYPNLKINNGVLYGEELVTLNISSKFKYFINRIQTLNGKHFIYFSNSTYGGLVIKYIMLSNGYSEYNGSQGTNPHMINGKPKTFAIVTSKMKSSLEDLLDVYNSPENDDGSQLMFLFSSNIMSESYTLKEVRHIWFMTIPDTFSQYNQILGRSIRKFSYADISEPVNVYLLAAVYSDFNDEVTSLNDYTQDELINVLPFDIKKLLYLKFKTKETNRIYSILQEMSETYSLPPHPSIVKVLLGELVRQFFYNNSRIKYNDSKLLKMVTSVIKNKEDARNYIDDIVNGHFFVSNKVFDKSLLYKYENDIITVPFRLSYEPFVWGVNFRKEYNVVSSP

SEQ ID NO: 3 - protein, 710 aa, Vaccinia virus
MRYIVSPQLVLQVGKGQEVERALYLTPYDYIDEKSPIYYFLRSHLNIQQPEIVKRHILLTLRMTQLKGYLGNLLDIKDDIIIYSHKNNLEYSYVDNTIFNPFVYTQKKTLLKNDSFLYNVYPGACDFLVIWVARACDTSIPEFGSYEDVDNNIIKFETMLMEVFPQLDLDITVESKFNNIFRTNLKLTGLKKIIQRVQDLDINYKSLLSRYDEHFINMTGNHFILNDEQLNLSIWDLDGTLALSSDGDTVMINNVKLFTDLVSDIDTQMERIKGDITYKVHLATPINSRIKLDIETSFIFIETATNNILLSSDKKISIILAKNHISIKVKNHIPNIEKYFTFLVIAINAMFNSVQKSADFTKVETVYWSRICQNTKNKNRKPIIINYLDPGMKKISNNFYKSDEKEVFINDNGIMFTCMDPLGKYNKVGFLNIFHDMRKYCIPCCFLHDQSHRSTFSSCVHQIDVEKKIVSPYILNFGKVVTESKMSFLPIIFDAFLNDGMTANMEQDNKRLKETSGYHIVRCCAGDDIVRLRTTSDIIQFVNEDKNILIVNDMVYFPMNASDIGKKIHILIQEIVHEVMIVKKKESSDKIDFFPPNYKLLKDLFPKQTIQTPIQSDAGMVLTTDGFYIDGKLFNEDLSSKYVTFTKNVIASDAVAKYFSPLFKYVISEAKDRFIKTWMINIMIHMNVDPNNIIPTLEKYYPNSGRAQIN

SEQ ID NO: 4 - protein, 101 aa, Vaccinia virus
MNSHFASAHTPFYINTKEGRYLVLKAVKVCDVRTVECEGSKASCVLKVDKPSSPACERRPSSPSRCERMNNPGKQVPFMRTDMLQNMFAANRDNVASRLLN

SEQ ID NO: 5 - protein, 644 aa, Vaccinia virus
MEAVVNSDVFLTSNAGLKSSYTNQTLSLVDEDHIHTSDKSLSCSVCNSLSQIVDDDFISAGARNQRTKPKRAGNNQSQQPIKKDCMVSIDEVASTHDWSTRLRNDGNAIAKYLTTNKYDTSNFTIQDMLNIMNKLNIVRTNRNELFQLLTHVKSTLNNASVSVKCTHPLVLIHSRASPRIGDQLKELDKIYSPSNHHILLSTTRFQSMHFTDMSSSQDLSFIYRKPETNYYIHPILMALFGIKLPALENAYVHGDTYSLIQQLYEFRKVKSYNYMLLVNRLTEDNPIVITGVSDLISTEIQRANMHTMIRKAIMNIRMGIFYCNDDDAVDPHLMKIIHTGCSQVMTDEEQILASILSIVGFRPTLVSVARPINGISYDMKLQAAPYIVVNPMKMITTSDSPISINSKDIYSMAFDGNSGRVVFAPPNIGYGRCSGVTHIDPLGTNVMGSAVHSPVIVNGAMMFYVERRQNKNMFGGECYTGFRSLIDDTPIDVSPEIMLNGIMYRLKSAVCYKLGDQFFDCGSSDIFLKGHYTILFTENGPWMYDPLSVFNPGARNARLMRALKNQYKKLSMDSDDGFYEWLNGDGSVFAASKQQMLMNHVANFDDDLLTMEEAMSMISRHCCILIYAQDYDQYISARHITELF

SEQ ID NO: 6 - protein, 273 aa, Vaccinia virus [sequence omitted]

SEQ ID NO: 7 - DNA, 19 bp, Artificial Sequence
  tetO2 sequence
tccctatcagtgatagaga

SEQ ID NO: 8 - DNA, 72 bp, Artificial Sequence
  PiA3L Promoter
ataagattggatattaaaatcacgctttcgagtaaaaactacgaatataaatatccctatcagtgatagaga

SEQ ID NO: 9 - DNA, 61 bp, Artificial Sequence
  Pi11 promoter
atatagtagaatttcattttgtttttttctatgctataaatatccctatcagtgatagaga

SEQ ID NO: 10 - DNA, 62 bp, Artificial Sequence
  PiD6R promoter
atatatgctcatatatttatagaagatatcacatatctaaatatccctatcagtgatagaga

SEQ ID NO: 11 - DNA, 141 bp, Artificial Sequence
  PiE8 promoter
gtataatcccattctaatactttaacctgatgtattagcatcttattagaatattaacctaactaaaagacataacataaaaactcattacatagttgataaaaagcggtaggatataaatatccctatcagtgatagaga

SEQ ID NO: 12 - DNA, 4029 bp, Artificial Sequence
  tetA, tetR gene
ctaaaatagcgctagagcaagctggactgttagtacttgcgatccggacaccattaattcgtaagttaaaacaggaaaaaccgggggaacttggcgaaatagcacgagtattggcggagaataacattaatattttagtgcaatacagtgaccatgctaaccaactgatattaataacggacaatgatagtatggctgcatctgttacgctcccttgggcaataaagtgaacttgcgatggctaatttaatacgaaaagaggttacctttgagtcctcaatagccgcgatagggggctcatgtctgacatttcacgagttaaaatactcagtgctttgatggatgggcgagcttggacggccactgagctaagttctgtggcgaatatatcagcttcaacggcgagcagtcatttatctaaattattagattgccagctaatcacagtagtagctcaaggcaagcatcgttattttcggctagcaggaaaagatattgctgaattgatggaaagtatgatggggatctccttaaaccatggcgtacatgccaaagtttccacgccagtgcatttacgaaaagcacgtacttgctatatgatcatttagctggcgaagttgccgttaagatctatgattccctttgtcaacagcaatggatcactgaaaatggttcaatgatcacattaagtggtattcaatattttcatgaaatgggaattgacgttccttccaaacattcacgtaaaatctgttgtgcgtgtttagattggagtgaacgccgtttccatttaggtgggtacgttggagccgcattattttcgctttatgaatctaaagggtggttaactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgaatagttcgacaaagatcgcattggtaattacgttactcgatgccatggggattggccttatcatgccagtcttgccaacgttattacgtgaatttattgcttcggaagatatcgctaaccactttggcgtattgcttgcactttatgcgttaatgcaggttatctttgctccttggcttggaaaaatgtctgaccgatttggtcggcgcccagtgctgttgttgtcattaataggcgcatcgctggattacttattgctggctttttcaagtgcgctttggatgctgtatttaggccgtttgctttcagggatcacaggagctactggggctgtcgcggcatcggtcattgccgataccacctcagcttctcaacgcgtgaagtggttcggttggttaggggcaagttttgggcttggtttaatagcggggcctattattggtggttttgcaggagagatttcaccgcatagtcccttttttatcgctgcgttgctaaatattgtcactttccttgtggttatgttttggttccgtgaaaccaaaaatacacgtgataatacagataccgaagtaggggttgagacgcaatcgaattcggtatacatcactttatttaaaacgatgcccattttgttgattatttatttttcagcgcaattgataggccaaattcccgcaacggtgtgggtgctatttaccgaaaatcgttttggatggaatagcatgatggttggcttttcattagcgggtcttggtcttttacactcagtattccaagcctttgtggcaggaagaatagccactaaatggggcgaaaaaacggcagtactgctcgaatttattgcagatagtagtgcatttgcctttttagcgtttatatctgaaggttggttagatttccctgttttaattttattggctggtggtgggatcgctttacctgcattacagggagtgatgtctatccaaacaaagagtcatgagcaaggtgctttacagggattattggtgagccttaccaatgcaaccggtgttattggcccattactgtttactgttatttataatcattcactaccaatttgggatggctggatttggattattggtttagcgttttactgtattattatcctgctatcgatgaccttcatgttaacccctcaagctcaggggagtaaacaggagacaagtgcttagttatttcgtcaccaaatgatgttattccgcgaaatataatgaccctcttgataacccaagagggcattttttacgataaagaagatttagcttcaaataaaacctatctattttatttatctttcaagctcaataaaaagccgcggtaaatagcaataaattggccttttttatcggcaagctcttttaggtttttcgcatgtattgcgatatgcataaaccagccattgagtaagtttttaagcacatcactatcataagctttaagttggttctcttggatcaatttgctgacaatggcgtttaccttaccagtaatgtattcaaggctaattttttcaagttcattccaaccaatgataggcatcacttcttggatagggataaggtttttattattatcaataatataatcaagataatgttcaaatatactttctaaggcagaccaaccatttgttaaatcagtttttgttgtgatgtaggcatcaatcataattaattgctgcttataacaggcactgagtaattgttttttatttttaaagtgatgataaaaggcacctttggtcaccaacgcttttcccgagatctcatctattgaaacagcttgatagcctttttcaacaaacaatattcgtgctgagttaaccagtgattgataggtactcttaaaattttcttgttgatgatttttattttccatgatagatttaaaataacataccgtcagtatgtttatggtatcatgatgatgtggtcgtgacaatcttaagaacatttaggttattttatgtatattgaacagcattctcgctatcaaaataaagctaataacatccaattagaatatgatgatagacagtttcatacaacggttatcaaagatgttctattatggattgaacataatttagatcagtctttactgcttgatgatgtggcgaataaagcgggttataccaagtggtattttcagcggctgttcaaaaaagtaacaggggtcacactggctagctatattcgtgctcgtcgtttgacgaaagcggctgttgagttgaggttgacgaaaaaaactatccttgagatcgcattaaaatatcaatttgattcccaacaatcttttacacgtcgatttaagtacatttttaaggttacaccaagttattatcggcgtaataaattatgggaattggaggcaatgcactgagagatc

(19) United States
(12) Patent Application Publication
(10) Pub. No.: US 2013/0171189 A1
(43) Pub. Date: Jul. 4, 2013

(54) INDUCIBLE AND REPRESSIBLE VACCINIA VIRUSES WITH IMPROVED SAFETY

(71) Applicants: Paulo Henrique Verardi, Mansfield Center, CT (US); Allison Titong, Vernon, CT (US); Caitlin Jeanette Hagen, Boston, MA (US); Brittany Jasperse, Brick, NJ (US)

(72) Inventors: Paulo Henrique Verardi, Mansfield Center, CT (US); Allison Titong, Vernon, CT (US); Caitlin Jeanette Hagen, Boston, MA (US); Brittany Jasperse, Brick, NJ (US)

(73) Assignee: UNIVERSITY OF CONNECTICUT, Farmington, CT (US)

(21) Appl. No.: 13/652,015

(22) Filed: Oct. 15, 2012

Related U.S. Application Data
(60) Provisional application No. 61/547,152, filed on Oct. 14, 2011.

(51) Int. Cl. A61K 39/285

(52) U.S. Cl. CPC A61K 39/285
USPC 424/199.1; 435/235.1; 435/236

(57) ABSTRACT

Described herein are recombinant vaccinia viruses that provide the efficacy of the current smallpox vaccine, but with a built-in safety mechanism, giving the physician or vaccine recipient (vaccinee) control over the vaccine virus replication. Specifically, genetic elements of the tetracycline (tet) operon and modified tet repressor genes are used to control the expression of vaccinia virus genes that are essential for viral replication, thereby allowing replication of the virus to be accurately regulated through the addition or removal of antibiotics (tetracyclines). The recombinant vaccinia viruses can be used as safer next-generation smallpox vaccines, as expression vectors for exogenous genes such as those encoding therapeutic or toxic proteins, as oncolytic viruses, and for tumor imaging and vector tracking in vivo.

### CROSS-REFERENCE TO RELATED APPLICATIONS

This application claims priority to U.S. Provisional Application 61/547,152 filed on Oct. 14, 2011, which is incorporated herein by reference in its entirety.

### FIELD OF THE DISCLOSURE

The present disclosure relates to modified vaccinia viruses with improved safety useful as next-generation vaccines for smallpox and as vectors for recombinant human and animal vaccines, immunotherapies, and oncolytic therapies.

### BACKGROUND

The use of vaccinia virus (VACV) as the smallpox vaccine was responsible for the eradication of smallpox, declared in 1980 by the World Health Organization (WHO). Vaccination with VACV, a poxvirus, provides protective immunity against variola virus, a related poxvirus and the causative agent of smallpox. However, the use of live-replicating VACV as the vaccine for smallpox can also cause a number of mild, moderate, or severe adverse reactions. The complications associated with the current vaccine make mass vaccination unfeasible in the United States, where an estimated 25% of the population is unable to receive the vaccine due to contraindications.

With the current smallpox vaccine, if serious adverse reactions occur, vaccinia immunoglobulin (VIG) and investigational new drugs such as cidofovir can be used (“off-label” for cidofovir) to help manage the complications. Although there is evidence from the smallpox eradication period that suggests individuals with adverse reactions benefit from VIG treatment, there have been no controlled studies. Intravenous VIG (VIGIV) was licensed for use in 2005 for the treatment of progressive vaccinia, eczema vaccinatum, severe generalized vaccinia and extensive body surface involvement or periocular implantation. Side effects associated with VIG treatment, although typically mild, can include severe events such as hypotension, renal dysfunction, and aseptic meningitis syndrome. Cidofovir is only recommended to treat adverse events if VIG treatment fails or if the patient is close to death. Side effects associated with cidofovir include renal toxicity, neutropenia, and metabolic acidosis Animal studies have also shown cidofovir to be carcinogenic.

Due to the large population at risk for developing adverse reactions and the lack of safe and effective treatments for such events, new and safer smallpox vaccines are in great need. New generation smallpox vaccines all focus on increasing vaccine safety while ideally maintaining efficacy. Many new generation vaccines have taken the approach of attenuating the vaccine virus. Such vaccines include MVA and NYVAC. NYVAC was developed by deleting 18 VACV genes, leaving the virus unable to replicate in humans. MVA (modified vaccinia Ankara) was generated by passing VACV in chick embryo fibroblasts (>570 times) until the virus lost the ability to replicate in most mammalian cells. This virus has been studied extensively as a safer alternative to replication-competent VACVs. Since MVA cannot replicate in human cells, no serious adverse reactions have been associated with it. However, even though MVA was used during the eradication period, it was never administered in an area where smallpox was endemic, and therefore the efficacy of MVA against variola has never been evaluated. While MVA is undisputedly safer than replication-competent smallpox vaccines, it has been shown to require multiple doses to achieve immunogenicity equivalent to Dryvax® (the original smallpox vaccine used in the U.S. during the smallpox eradication campaign) or ACAM2000 (the currently licensed smallpox vaccine in the U.S., derived from Dryvax®).

Unfortunately, the use of a replication-deficient smallpox vaccine requiring a two or more dose regimen is not ideal in a bioterrorism event involving smallpox. One suggested approach for the use of MVA or other replication deficient VACVs is to immunize before there is any risk of smallpox infection and boost with the standard smallpox vaccine or with MVA in the event of an immediate threat. This proposed vaccination scheme would require keeping the population vaccinated with MVA in preparation for a possible smallpox threat.

The U.S. Food and Drug Administration (FDA) has suggested that the regulatory considerations for licensure of next-generation smallpox vaccines will likely include (1) demonstration of immunologic non-inferiority to ACAM2000® (the current licensed vaccine) in clinical studies, and (2) demonstration of efficacy in animal models using related orthopoxviruses (aka the “animal rule”). The only known correlate of protection for smallpox is the scarring that results from intradermal inoculation by scarification, referred as the “take”. Dryvax® and ACAM2000® produce the take, while the highly attenuated strains of VACV such as NYVAC and MVA do not.

VACV has also been used extensively as a vector for the development of recombinant human and animal vaccines, as well as immunotherapeutic vaccines and oncolytic virotherapies for cancer. The use of live replicating VACV vaccine vectors is highly desirable because replication-competent viruses elicit stronger immune responses than highly attenuated or non-replicating viruses, and require smaller doses for administration. Moreover, oncolytic virotherapies to treat cancers (either alone or in combination with other therapies) require fully-replicating viruses. These replicating VACV vectors can also cause adverse reactions in vaccinees, vaccinators, and contacts. This is particularly relevant to cancer patients, which typically have some level of immunosuppression.

What is needed are vaccines with a built-in safety mechanism that would promote the wide use of live replicating VACV vectors for vaccines and therapeutics.

### SUMMARY

In one aspect, a recombinant VACV comprises a first recombinant nucleic acid comprising a tetracycline (tet) response element and a nucleic acid encoding a conditional replication gene product, wherein the tet response element is operably linked to the nucleic acid encoding the conditional replication gene product; and a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor; wherein the second recombinant nucleic acid is located in a non-essential region of the VACV genome or in an intergenic region of the VACV genome, and wherein the conditional replication gene product is a VACV gene product essential for virus replication, wherein the essential VACV gene product is not the A14 protein, and wherein expression of the conditional replication gene product is inducible or repressible by a tetracycline (TC) antibiotic.

In another aspect, a method of vaccinating an individual against smallpox comprises administering to the individual the VACV described above in an amount sufficient to elicit an immune response.

In an additional aspect, the recombinant VACV further comprises an exogenous gene operably linked to an expression control sequence for the exogenous gene, wherein the product of the exogenous gene is a therapeutic protein such as an antigen. Such recombinant VACVs can be used to vaccinate or treat an individual.

In a yet further aspect, a recombinant vaccinia virus comprises a first recombinant nucleic acid comprising a tet response element and a nucleic acid encoding a conditional replication gene product, wherein the tet response element is operably linked to the nucleic acid encoding the conditional replication gene product; and a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor, and wherein the second recombinant nucleic acid is located in a non-essential region of the vaccinia virus genome or in an intergenic region of the vaccinia virus genome; wherein the conditional replication gene product is a gene product that is toxic to a mammalian host or cells, lowers the fitness of the recombinant vaccinia virus, or interferes with the replication of VACV, and wherein expression of the conditional replication gene product is inducible or repressible by a tetracycline antibiotic.

In a yet further aspect, a method of making a high titer virus preparation of a highly-attenuated vaccinia virus comprises culturing the recombinant vaccinia virus of claim 1 in a medium comprising a tetracycline antibiotic, purifying the recombinant vaccinia virus in the absence of a tetracycline antibiotic to produce a recombinant vaccinia virus preparation, and titering the recombinant vaccinia virus preparation in the presence of a tetracycline antibiotic.

### BRIEF DESCRIPTION OF THE FIGURES

FIG. 1 is a schematic of a replication-inducible VACV. In the absence of TCs (denoted by stars), TetR protein (hexagons) is produced and binds to the operator sequence in the promoter (P_{O2}) for the essential VACV gene, preventing transcription and therefore virus replication. In the presence of TCs, TC binds TetR and prevents TetR from interacting with the operator, thus allowing transcription of the essential gene and virus replication.

FIG. 2 is a schematic of the genetic setup for replication-inducible (panel A) and replication-repressible (panel B) VACVs tested.

FIG. 3 shows the effect of doxycycline (DOX) concentration on plaque size for recombinant viruses expressing essential VACV genes inducibly under the control of tet operon elements. The effect of DOX concentration on plaque radius is depicted. BS-C-1 cells were infected with 40 plaque-forming units (PFU)/well of virus and allowed to grow in the presence of 0, 1, 10, 100, or 1000 ng/ml DOX for 40 hr. At 40 hr plaque sizes were measured. Error bars=standard error of the mean (SEM). ***=p<0.0001 (paired t-test). AB=abortive infections.

FIG. 4 shows the effect of DOX on plaque formation for selected replication-inducible VACVs. BS-C-1 cell monolayers were infected with wild-type VACV (WR), viA7L, or viD6R in the presence (+) or absence (−) of 1 μg/ml DOX. The cells were stained with crystal violet at 48 hr post-infection (middle row of wells) or 7 days post-infection (bottom row of wells).

FIG. 5 shows the effect of different TCs on plaque formation. BS-C-1 cells were infected with 20 PFU/well of the virus and allowed to grow in the presence of 1 μg/ml DOX, tetracycline (TET), or anhydrotetracycline (ATC), or no TCs. No significant differences were observed among the different TCs. Error bars=SEM. AB=abortive infections.

FIG. 6 shows the effect of DOX on viral replication for selected replication-inducible VACVs. BS-C-1 cell monolayers were infected with 0.01 PFU of WR, viA7L, or viD6R per cell in the presence (+) or absence (−) of 1 μg/ml DOX in triplicates. The cells were collected at the indicated times after infection and viral titers were determined by plaque assay in the presence of 1 μg/ml DOX.

FIG. 7 shows viP11A3L isolates tested in the presence and absence of DOX. In the presence of DOX, isolates produced between 50 and 80 fluorescent plaques. In the absence of DOX, no fluorescent plaques were observed. However, abortive infections (single fluorescent cells) were observed for all three isolates in the absence of DOX.

FIG. 8 shows viP11E8R isolates tested in the presence and absence of DOX. In the presence of DOX, isolate #111 and #223 produced 66 and 100 fluorescent plaques, respectively. In the absence of DOX, isolate #111 formed 139 fluorescent plaques while isolate #223 formed 74 plaques. Isolate #112 produced 9 fluorescent plaques in the presence of DOX and 13 in the absence of DOX.

FIG. 9 shows the construction of the transfer vector pAT020 for the generation of vRG0. The cloning steps leading to the generation of the final transfer vector are shown. pAT020 contains the gpt-gus fusion gene under the control of a synthetic early/late VACV promoter (P_{Sel}), the EGFP gene under the control of an engineered late P₁₁ promoter with a tetO₂ sequence located directly downstream, the revtetR gene (L17G) under the control of a constitutive strong synthetic early/late promoter (P_{E/L}), all flanked by thymidine kinase (TK) left (TK_{L}) and right (TK_{R}) sequences that direct recombination with the TK gene of VACV.

FIG. 10 shows that EGFP expression is repressed in the presence of increasing concentrations of ATC when measured by a multiwell fluorescence reader. In this preliminary study, BS-C-1 cells were infected at a multiplicity of infection (MOI) of 1 with the VACVs in the absence or presence of ATC and 2 days post-infection cells were fixed and quantified with a fluorometer. EGFP expression levels were repressed in the presence of increasing concentrations of ATC. Error bars are SEM, n=2.

FIG. 11 shows that EGFP expression is repressed in the presence of increasing concentrations of TET when measured by a multiwell fluorescence reader. In this preliminary study, BS-C-1 cells were infected at an MOI of 1 with the VACVs in the absence or presence of TET and 2 days post-infection cells were fixed and quantified with a fluorometer. EGFP expression levels were repressed in the presence of increasing concentrations of TET. Error bars are SEM, n=2.

FIG. 12 shows that EGFP expression is repressed in the presence of increasing concentrations of DOX when measured by a multiwell fluorescence reader. In this preliminary study, BS-C-1 cells were infected at an MOI of 1 with the VACVs in the absence or presence of DOX and 2 days post-infection cells were fixed and quantified with a fluorometer. EGFP expression levels were repressed in the presence of increasing concentrations of DOX. Error bars are SEM, n=2.

FIG. 13 shows that ATC represses EGFP expression in a dose response manner. BS-C-1 cells were infected at an MOI of 1 with the VACVs in the absence or presence of ATC and 2 days post-infection cells were fixed and quantified with a multiwell fluorescence reader. EGFP expression levels were repressed in the presence of increasing concentrations of ATC. Error bars are SEM, n=2.

FIG. 14 shows that TET represses EGFP expression in a dose response manner. BS-C-1 cells were infected at an MOI of 1 with the VACVs in the absence or presence of TET and 2 days post-infection cells were fixed and quantified with a multiwell fluorescence reader. EGFP expression levels were repressed in the presence of increasing concentrations of TET. Error bars are SEM, n=2.

FIG. 15 shows that DOX represses EGFP expression in a dose response manner. BS-C-1 cells were infected at an MOI of 1 with the VACVs in the absence or presence of DOX and 2 days post-infection cells were fixed and quantified with a multiwell fluorescence reader. EGFP expression levels were repressed in the presence of increasing concentrations of DOX. Error bars are SEM, n=2.

FIG. 16 is a schematic of a replication-repressible VACV. In the absence of TCs (denoted by triangles), RevTetR protein (circles) is produced but does not bind to the tetO₂ operator sequence in the promoter (P₁₁) for the essential VACV gene (A6L), allowing transcription and virus replication. In the presence of TCs such as DOX, RevTetR binds TC and is then allowed to interact with the operator, thus preventing transcription and virus replication.

FIG. 17 shows preliminary results that indicate that viral replication is repressed in the presence of increasing concentrations of DOX in some of the repressible VACVs controlling expression of the essential A6L gene. BS-C-1 cells were infected with vRG1A6L-vRG5A6L (80-100 PFU/well) in the absence or presence of increasing concentrations of DOX and imaged 2 days post-infection. Replication was repressed in the presence of increasing concentrations of DOX for vRG2A6L, vRG3A6L, and vRG4A6L.

### DETAILED DESCRIPTION

In one embodiment, the recombinant VACVs described herein provide the efficacy of the current smallpox vaccine, but with a built-in safety mechanism, giving the physician or vaccine recipient (vaccinee) control over the vaccine virus replication. With these improved VACVs, the entire population in a given geographical area can be vaccinated against smallpox knowing that any complications resulting from vaccination can be either stopped (allowing the innate and adaptive immune system to destroy residual virus) or avoided altogether.

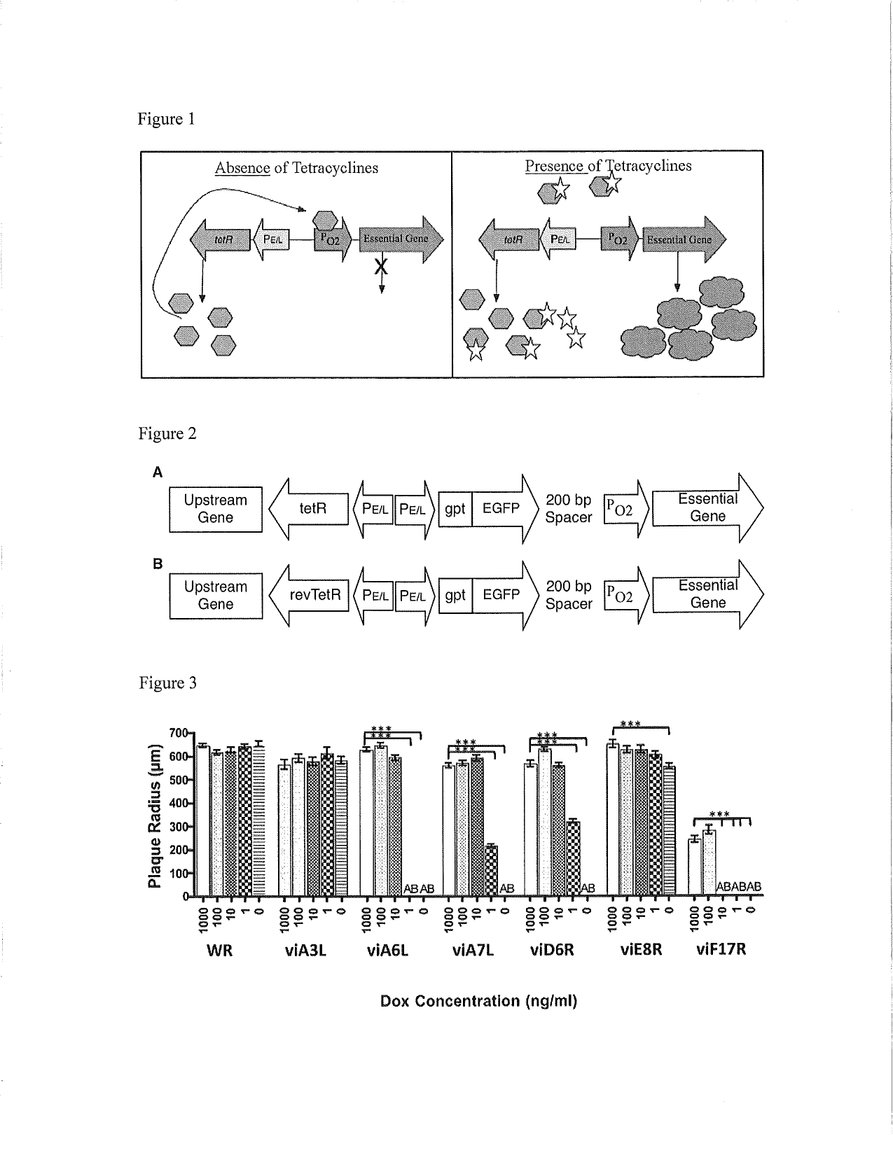

In one embodiment, described herein is the use of genetic elements of the tetracycline (tet) operon and modified tet repressor genes to control the expression of VACV genes that are essential for viral replication, thereby allowing replication of the virus to be accurately regulated through the addition or removal of antibiotics (TCs). Specifically, regulation of an essential gene can be achieved by constitutively expressing the tet repressor protein (TetR) within the VACV genome and incorporating a tet operator (tetO) into the promoter of an essential gene, thus making virus replication dependent on TCs. TC-induced gene expression in VACV is illustrated schematically in FIG. 1. In the absence of TCs (denoted by star), TetR protein is produced and binds to the operator sequence in the promoter for the essential VACV gene, preventing transcription and virus replication. In the presence of TCs, TC binds TetR and prevents TetR from interacting with the operator, thus allowing transcription of the essential gene and virus replication. In specific embodiments, the recombinant vaccinia virus is replication competent either in the presence or absence of tetracycline antibiotics.

These technologies are useful for the production of safer (1) next-generation smallpox vaccines, (2) recombinant human and animal vaccines, (3) immunotherapeutic vaccines, and (4) oncolytic virotherapies. In addition, these technologies are also useful to produce inducible and repressible stable VACV vectors that express gene products that are toxic to host cells, that reduce the overall fitness of the virus, or that interfere with the replication of VACV. Such genes products tend to be deleted or mutated by selective pressure (thus making the recombinant virus genetically unstable), or to cause a decrease in viral titers (yield) in cell culture or viral replication in vivo. Examples of such genes include exogenous viral genes that interfere with VACV replication or that are toxic to cells, such as the human immunodeficiency virus (HIV) envelope glycoprotein or the vesicular stomatitis virus G glycoprotein, and bacterial toxins.

VACV has been used as a vector for the development of human and animal vaccines expressing foreign antigens from disease-causing pathogens, such as the sylvatic rabies and the rinderpest vaccines for animals. These live-replicating VACV-based vaccines can also cause a number of significant adverse reactions in human vaccinees, or in the case of animal vaccines, personnel that administer the vaccine (vaccinators) or individuals that have contact with vaccinated animals, exposed vaccinators, or vaccine baits. The development of new VACV-based vaccines with the safety mechanisms described in this disclosure would be highly beneficial in these situations.

VACV has also been developed as a vector for the expression of tumor antigens such as the prostate specific antigen (PSA) for cancer treatment (immunotherapies), and more recently as oncolytic vectors for cancer therapy. As a replication-competent virus, VACV displays a natural tumor tropism and kills cancer cells by apoptosis and other mechanisms. Systemic administration of VACV (e.g., intravenous) targets both tumors and metastases, and clinical trials suggest that VACV can be an effective oncolytic cancer therapy for a variety of different cancers. Since cancer patients typically have some level of immunosuppression and are at higher risk for significant adverse reactions, the technologies described in this application would advance the use of such therapies in these targeted populations by allowing the safer use of replication-competent VACV therapeutic vectors.

Lactose (lac) and tet operon elements have been used previously to control the expression of genes in VACV. The use of the tet operon in VACV has previously focused on investigating the specific functions of genes. Many of the VACV genes described herein have not yet been placed under the control of tet operon elements, and the A6L gene has not previously been successfully controlled through the use of an operon system (either the lac or tet operons).

The use of mutations in the tetR gene to reverse the phenotype of TetR in the tet operon system was recently adapted to VACV by the inventors. These previously-characterized mutations of TetR generate a number of reverse TetR (RevTetR) proteins that only bind to the operator in the presence of TCs, making the operon system repressible. This repressible tet operon system has not previously been adapted to VACV.

As described herein, the utilization of both the tet operon elements and the reverse TetR mutations to control viral replication through conditional expression of VACV essential genes is a unique approach to improving the safety of smallpox vaccines and VACV vectors. This approach allows the vaccine to be tailored to an individual's contraindication. For example, the inducible vaccine may be used in the event that an individual with no contraindications requires the vaccine, but has close contacts with contraindications. This can be illustrated by a case of inadvertent inoculation that occurred in Indiana in 2007 when a U.S. service member received smallpox vaccination and then visited his family. H is son (who had eczema), developed a life-threatening case of eczema vaccinatum (a severe complication from VACV infection) from contact with his father. The boy required investigational antivirals, VIGIV, and 48 days of hospitalization to recover. If the inducible VACV had been used, inadvertent inoculation would have been avoided altogether, as TCs would need to be taken with the vaccine for viral replication to occur. As long as the child (contact) was not taking TCs, the virus would not have been able to replicate and eczema vaccinatum would not have developed.

An example where the repressible vaccine may be used is for the oral rabies vaccine, which is composed of a VACV vaccine vector expressing the rabies glycoprotein and is used across the U.S. and Europe to vaccinate wildlife. The vaccine baits are composed of a plastic packet containing the vaccine and coated in fishmeal to attract animals. Once an animal bites the bait the packet of vaccine is broken and the vaccine leaks into the mouth, resulting in viral replication and immunization of the animal. Although oral rabies vaccination programs are careful of where baits are dropped, inevitably people come in contact with the baits and the VACV vaccine vector they carry. One such incident occurred in Pennsylvania in 2009 when a dog brought its owner a ruptured vaccine bait. The owner had cuts on her hands and developed a VACV infection from handling the bait. As the owner was on immunosuppressive medication for inflammatory bowel disease, treatment with VIGIV and investigational antiviral agents were required to clear the VACV infection. However, she was not able to remain off her immunosuppressive medication for an extended period, making her treatment difficult. If a repressible VACV had been used in the rabies vaccine the woman could have been treated with TCs to stop the VACV infection and may not have needed to be removed from her immunosuppressive medication.

The repressible VACV may also be used to vaccinate non-contraindicated persons in case adverse reactions occur due to an unknown contraindication (for example undiagnosed HIV infection), or an entire segment of the population in the case of an emergency such as a smallpox bioterrorist event. TC antibiotics would then be administered at the first signs of any moderate to severe complication to stop viral replication, allowing resolution of the complication (e.g., eczema vaccinatum) without the need for VIG and investigational therapies.

Disclosed herein are vaccines that are replication competent, for example, made from the same parental VACV strain as the current (e.g., ACAM2000®) or past (e.g., Dryvax®) licensed vaccines, and are expected to induce protective immune responses non-inferior to the current licensed smallpox vaccine. The vaccines contain a built-in safety mechanism, allowing replication of the virus (that ultimately can lead to adverse reactions) to be stopped by either administering or withdrawing antibiotics (TCs). This regulation of virus replication is achieved by placing VACV genes that are essential for virus replication under the control of tet operon elements. The inventors made several inducible and repressible recombinant viruses using the built-in tet operon safety mechanism for proof-of-concept experiments using the Western Reserve (WR) strain of VACV.

Exemplary strains of VACV that would be used for vaccine and therapeutic applications include but are not limited to Dryvax®, ACAM2000®, and other New York City Board of Health (NYCBH) derived strains, Lister, Tian Tan, Copenhagen, and the like.

The tet operon system was first adapted to VACV to investigate the function of A14, a membrane protein, during VACV life cycle. The transposon Tn10 tet operon, in its natural E. coli host genome, consists of two genes, the resistance gene (tetA) and the repressor gene (tetR). The tetR gene produces a repressor protein (TetR) that binds to the tet operator sequences (tetO) that overlap tet operon promoters, thus inhibiting the transcription of the tet operon genes (tetA and tetR). TetR binds to TCs such as tetracycline (TET), doxycycline (DOX), and anhydrotetracycline (ATC), altering its conformation so that it is unable to bind to the operator sequences, thus allowing transcription of the operon genes. The two binding sites (operators) for TetR in the tet operon (tetO₁ and tetO₂) consist of 19 bp sequences that bind two molecules of TetR (one homodimer). TetR binds to operator tetO₂ with three- to five-fold higher affinity than to operator tetO₁.

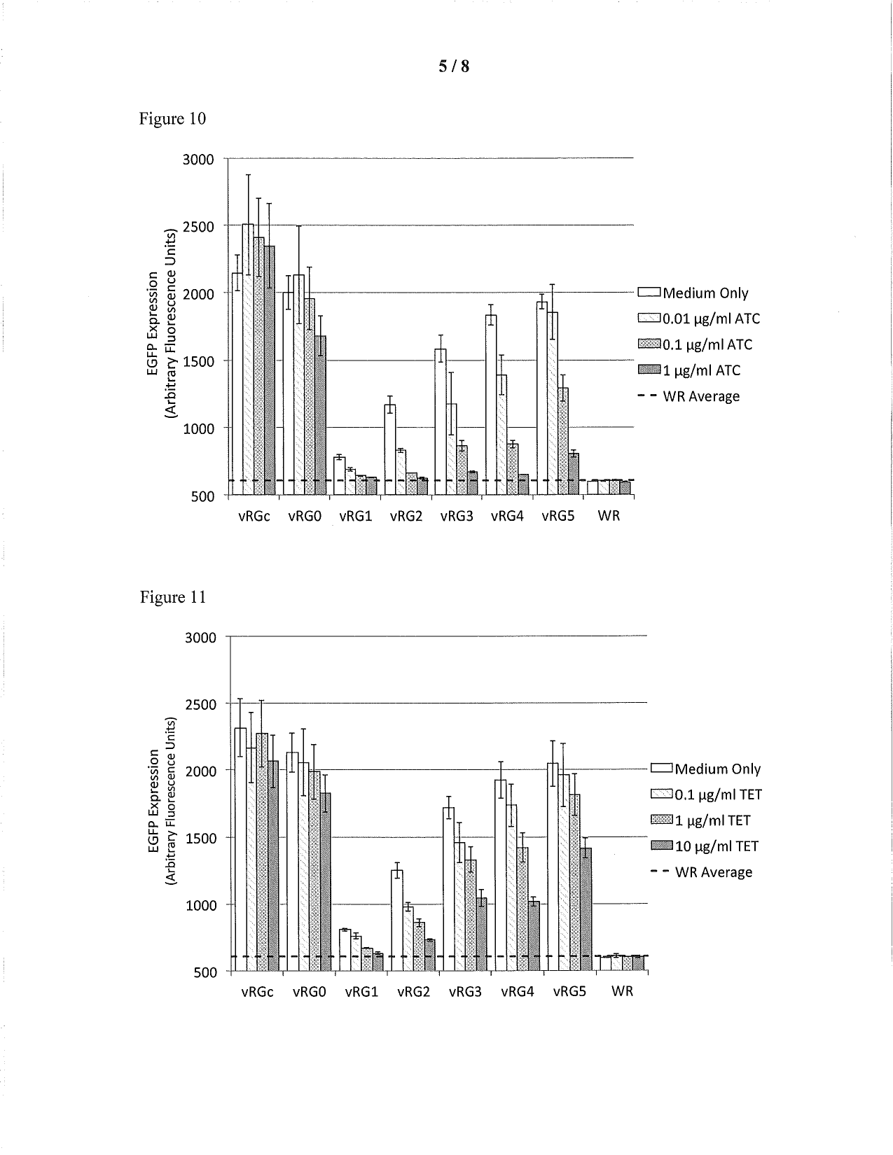

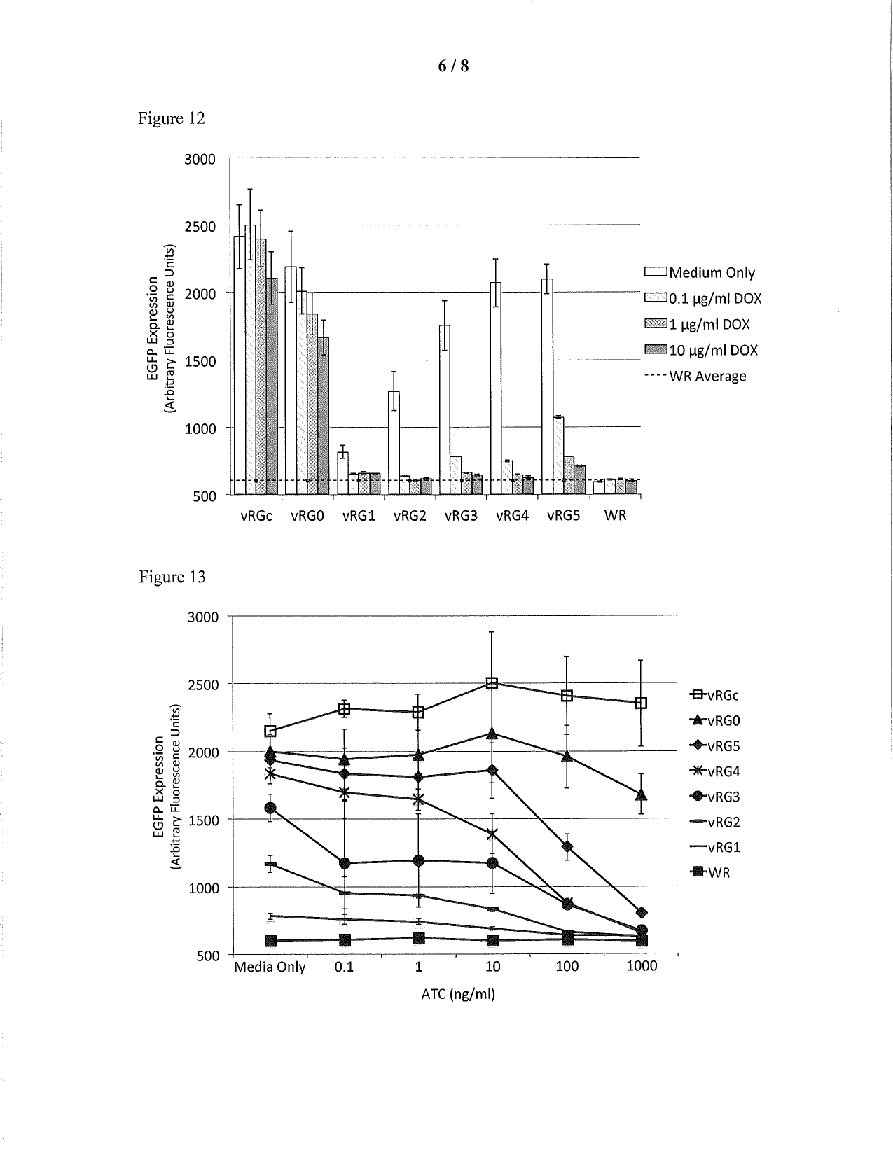

Mutagenesis studies have shown that the response of the TetR repressor can be reversed, causing TetR to act as an inducible repressor. A variety of single and multiple mutations in the tetR gene that are able to produce this phenotype have been described in mammalian and prokaryotic systems. The reverse form of the protein, RevTetR, is only able to bind to the operator sequence and block transcription in the presence of TCs. As shown in the Examples, five known RevTetR mutants produced the desired DOX dose response in VACV and can be used in the repressible VACV system (FIGS. 10-15).

In one embodiment, a recombinant VACV comprises a first recombinant nucleic acid comprising a tet response element and a nucleic acid encoding a conditional replication gene product, wherein the tet response element is operably linked to the nucleic acid encoding the conditional replication gene product; and a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor, and wherein the second recombinant nucleic acid is located in a non-essential region of the VACV genome or in an intergenic region of the VACV genome. The conditional replication gene product is an essential VACV gene product, wherein the essential VACV gene product is not the A14 protein. Further, expression of the conditional replication gene product is inducible or repressible by a TC antibiotic. In specific embodiments, the VACV is replication competent either in the presence or absence of TC antibiotics.

While an intergenic region can be a non-essential region of the vaccinia virus genome, intergenic regions can be, for example, promoters or other upstream and downstream regulatory elements. Further, while non-essential regions of the genome can include non-essential intergenic sequences, they can also include non-essential genes such as the TK gene, which is dispensable for virus replication, although the virus is attenuated. In one embodiment, the intergenic or non-essential region of the vaccinia virus genome is immediately upstream from the conditional replication gene.

As used herein, a tet response element is an element that is activated or repressed by a tet repressor or a reverse tet repressor that conditionally binds the tet response element. In one embodiment, a tet response element is a VACV promoter operably linked to a tet operator, such as the tetO₁ and tetO₂ operators. In one embodiment, the tet response element includes multiple tet operators, wherein the multiple tet operators are the same or different and are placed at variable distances from each other. In this embodiment, the use of multiple tet operators should allow fine tuning of repression or induction of the conditional replication gene product by allowing tighter binding of TetR or reverse TetR to the tet response element, resulting in lower levels of expression of the conditional replication gene under non-inducible conditions. In a specific embodiment, the tet response element comprises two or more tet operators.

As used herein, expression control sequences are promoters or transcription binding sites. Exemplary expression control sequences include poxvirus early/late promoters, poxvirus early promoters, poxvirus intermediate promoters, poxvirus late promoters, synthetic poxvirus promoters, tet response elements, and other inducible or constitutive promoters. The expression of the conditional replication gene product can also be modulated by varying the type and strength of the promoter used in the tet response element, allowing the fine tuning of repression or induction according to the desired application.

In one aspect, the tet response element (e.g., including a tet operator) is inserted between a transcriptional initiator of the conditional replication gene and a translational start site of the conditional replication gene, and optionally the second recombinant nucleic acid comprising an expression control sequence and a nucleic acid encoding a tet repressor or a reverse tet repressor is inserted into the viral genome in the intergenic region between the nucleic acid encoding the conditional replication gene product and its upstream gene. Placement of one or more tet operators (e.g., tetO₁, tetO₂) within or next to the expression control sequence (e.g., promoter) for the conditional replication gene product can be used to adjust induction/repression.

In one embodiment, wherein the tet response element overlaps the transcriptional initiator of the conditional replication gene.

The recombinant viruses generated in the examples utilize TetR or mutant forms of TetR (RevTetRs) and operator tetO₂ to control the expression of essential VACV genes. In the viral genome, the tetR gene (or revtetR) is under the control of an early/late constitutive VACV promoter (P_{E/L}) in the intergenic region between the essential gene and its upstream gene. Operator tetO₂ is inserted between the transcriptional initiator of the essential gene promoter and the translational start site of the essential gene to allow TetR (or RevTetR) to bind and block transcription. The sequence of the tetR gene can be found in Accession Number J01830.1 (SEQ ID NO. 12).

In a specific embodiment, the second nucleic acid is inserted into the viral genome in a way that does not substantially affect the viability of the virus.

The conditional replication gene product is an essential VACV gene product, wherein the essential VACV gene product is not the A14 protein. In specific embodiments, the essential VACV gene product is A6L, A7L, D6R, F17R, A3L, E8R, or a combination thereof. The sequences of the VACV proteins (WR strain) can be found in Accession Number NC-006998.1.

The gene A6L is one of 91 open reading frames (ORFs) conserved among all chordopoxviruses. The A6L gene product (SEQ ID NO. 1) is expressed late in infection, tightly packaged into the virion core and appears to be essential in virion morphogenesis. An inducible A6L recombinant had been attempted using the lac operon system, but was unsuccessful.

The A7L and D6R genes make up the VACV early transcription factor (VETF). The small subunit (70 kDa) is encoded by D6R (SEQ ID NO. 2) and the large subunit (80 kDa) is encoded by A7L (SEQ ID NO. 3) (referred to as A8L in some literature, ORF A8 is now considered to be A8R and to encode and intermediate transcription factor). Lac operon inducible recombinants have been made for both genes. In the absence of inducer both recombinants were defective in virion morphogenesis and mature virions were not frequently formed.

F17R (SEQ ID NO. 4), also referred to as F18 or p11, is one of the most abundant core proteins, accounting for 11% of the virion mass. F17R binds strongly to DNA and has been characterized as a DNA-binding protein. An inducible F17R recombinant has been generated using the lac operon system. In the absence of inducer the recombinant was unable to replicate and virion morphogenesis was blocked at an intermediate stage.

The A3L gene encodes the precursor to the virion core protein 4b. Proteolytic processing of the A3L gene product (SEQ ID NO. 5) is required for the formation of intracellular mature virus (IMV) during viral morphogenesis. Mutant 4b proteins hinder the necessary structural rearrangements needed for the transition to IMV and formation of the core wall, generating disorganized virions that are defective in transcription.

The specific role of the VACV E8R gene has not yet been accurately determined although several studies have been conducted. E8R was predicted to contain two transmembrane domains and was first investigated as a potential membrane protein involved in ER wrapping. E8R was shown to localize to DNA replication sites and to be concentrated in the ER surrounding the replication site. Based on these findings E8R was suggested to be an ER-resident membrane protein that may bind newly synthesized VACV DNA and aid in ER wrapping. The E8R gene product is SEQ ID NO. 6.

In another embodiment, the recombinant VACVs can be used as a vector for human and animal vaccines, as an immunotherapeutic vector, as an oncolytic vector (e.g., expressing cytokines), or as a vector to express toxic or unstable genes. The exogenous genes could be foreign antigens in the case of VACV-vectored recombinant vaccines for human and animals (e.g., expressing human papilloma, HIV, influenza, hepatitis B, rabies, Rift Valley fever, Newcastle disease, and rinderpest antigens), tumor antigens in the case of immunotherapeutic VACV vectors (e.g., expressing antigens such as prostate specific antigen or PSA, or carcinoembryonic antigen or CEA), genes that enhance the oncolytic potential of VACV in the case of oncolytic virotherapy (e.g., expressing cytokines such as GM-CSF or other immunostimulatory molecules), or other reporter genes that can be used for molecular in vivo imaging after systemic delivery for non-invasive detection of primary and metastatic tumors or to track the therapeutic vector during treatment (e.g., somatostatin receptor, luciferase, fluorescent proteins).

In one embodiment, the recombinant VACV further comprises an exogenous gene operably linked to an expression control sequence for the exogenous gene, wherein the product of the exogenous gene is a therapeutic protein. Exemplary exogenous genes include a foreign antigen from a human or an animal, a tumor antigen, or a gene that enhances the oncolytic potential of the recombinant VACV. In one embodiment, a method of vaccinating an individual comprises administering to the individual the recombinant VACV including an exogenous gene in an amount sufficient to elicit an immune response.

In another embodiment, the technology can be used to make recombinant VACVs that conditionally express a gene of interest, comprising an exogenous gene operably linked to an expression control sequence for the exogenous gene, wherein the product of the exogenous gene is toxic to a mammalian host or reduces the fitness of the recombinant VACV. These stable recombinant VACVs that express gene products that are toxic or interfere with VACV replication tend to be deleted, mutated, or they tend to cause a decrease in viral titers (yield) when passaged in cell cultures or in vivo. Under conditions that do not lead to expression of the gene product (e.g., a toxic gene product), the virus replicates in mammalian systems (cells, human, or animal hosts) with minimal selective pressure to delete, mutate, or otherwise stop the expression of that gene or genes of interest, or not interfering with viral replication and yield. Examples of such genes include exogenous viral genes such as the human immunodeficiency virus (HIV) envelope glycoprotein or the vesicular stomatitis virus G glycoprotein, and bacterial toxins.

In one embodiment, a recombinant vaccinia virus comprises a first recombinant nucleic acid comprising a tet response element and a nucleic acid encoding a conditional replication gene product, wherein the tet response element is operably linked to the nucleic acid encoding the conditional replication gene product; and a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor, and wherein the second recombinant nucleic acid is located in a non-essential region of the vaccinia virus genome or in an intergenic region of the vaccinia virus genome, and wherein the conditional replication gene product is a gene product that is toxic to a mammalian host or cells, lowers the fitness of the recombinant vaccinia virus, or interferes with the replication of VACV, and wherein expression of the conditional replication gene product is inducible or repressible by a tetracycline antibiotic.

In another aspect, the inducible recombinant VACVs disclosed herein could be used as the so called “third-generation” smallpox vaccines or vectors, since in vivo (upon vaccination) they would behave like highly-attenuated MVA and NYVAC VACVs (single cycle abortive infection with antigen expression), but could be grown and titered more easily in mammalian cell cultures under TC treatment. Attenuated viruses have reduced or no virulence, but are viable. For example, MVA and NYVAC do not grow in mammalian cell lines, particularly those approved for vaccine production, being able to replicate only in primary or secondary CEF chick embryo fibroblasts (CEF) or Syriam hamster kidney (BHK-21) cells. Maintaining CEF cultures is challenging, as they require egg material from pathogen-free chickens and only survive a few passages. In addition, MVA and NYVAC viral yields obtained in CEF or BHK-21 cells are low, and titration of the viral stocks requires labor-intensive immunostaining techniques, since the viruses do not form distinct plaques. In this sense inducible VACV vectors could also be used for the development of MVA-like human and animal vaccines and immunotherapies. As such, propagation of the virus would be performed in mammalian cell substrates that are FDA approved or that yield higher titers, and titration of the stocks could be performed by standard techniques. For production, TCs would be added to the cell culture to allow the inducible viruses to grow to higher titers as fully-replicating viruses, but vaccination or treatment of animals or human subjects would be carried out in the absence of TCs, causing an abortive infection (single round of infection) similar to MVA and NYVAC. This method could prove very useful in the vaccination of immunocompromised or immunosuppressed individuals, children, and the elderly.

Thus, in one embodiment, a method of making a high titer virus preparation of a highly-attenuated vaccinia virus, comprising culturing a recombinant vaccinia virus as described herein in a medium comprising a tetracycline antibiotic, purifying the recombinant vaccinia virus in the absence of a tetracycline antibiotic to produce a recombinant vaccinia virus preparation, and titering the recombinant vaccinia virus preparation in the presence of a tetracycline antibiotic.

The term “recombinant VACV” refers to a replication-competent VACV that includes at least one exogenous nucleic acid. Specifically, as used herein, a “recombinant VACV” includes at least two exogenous nucleic acids, specifically an exogenous nucleic acid encoding a tet repressor protein (or a reverse tet repressor protein), and a tet response element (e.g., tet operator sequence) that renders the VACV drug-sensitive or drug-dependent. In some embodiments, additional exogenous nucleic acids may be present that allow expression control sequences (e.g., poxvirus promoters) to express one or more exogenous genes constitutively, inducibly, or repressibly. The exogenous genes could be foreign antigens and immunostimulatory molecules in the case of VACV-vectored recombinant vaccines for human and animals, tumor antigens in the case of immunotherapeutic VACV vectors, genes that enhance the oncolytic potential of VACV in the case of oncolytic virotherapy, or other genes that can be used for non-invasive tumor imaging or vector tracking in vivo.

A “conditional replication gene product” refers to a gene product upon which continued existence of the VACV in the mammalian host environment depends. Viral existence may depend on, for example, replication of the viral genome, packaging of the viral genome, and expression of viral genes.

In one aspect, dependence on the conditional replication gene product may rely on the presence of an exogenous factor such as a drug. Thus, continued virus replication may depend on the presence of the conditional replication gene product and a drug, such that upon withdrawal of the drug, the conditional replication gene product is no longer produced. Alternatively, continued virus replication may be sensitive to the presence of a drug.

While variants of the tet repressor system may be used, the appended non-limiting examples refer to the classical system in which the repressor protein is bound to the tet response element in the absence of a TC antibiotic, thereby repressing transcription of the subject gene. Conversely, when drug is present, the tet repressor protein binds to the drug, not the tet repressor element, thereby removing the impediment to transcription. Nonlimiting examples of transcription repressors include the tet repressor, and the reverse tet repressor. The mutated or reverse TetR protein binds to the tet response element and suppresses transcription in the presence of DOX and other TCs.

Recombinant VACVs may be constructed by methods known in the art, and typically by homologous recombination. Standard homologous recombination techniques utilize transfection with DNA fragments or plasmids containing sequences homologous to viral DNA, and infection with wild-type or recombinant VACV, to achieve recombination in infected cells. Conventional marker rescue techniques may be used to identify recombinant VACV. Transient dominant selection may be used to develop some of the recombinants. In addition, more than one recombination step may need to be used to develop a recombinant VACV.

Recombination plasmids may be made by standard methods known in the art. The nucleic acid sequences of the VACV are known in the art. The VACV used for recombination may contain other deletions, inactivations, exogenous DNA, and genetic elements.

VACV strains are produced that are sensitive to a molecule based on the expression or non-expression of an essential gene which is operably linked to a tet response element when a nucleic acid encoding a tet repressor or a reverse tet repressor that conditionally binds the tet response element is under constitutive expression by an expression control sequence. In specific embodiments, the molecule selected is suitable for administration to humans and other subjects. Nonlimiting examples of TC antibiotics to which VACV strains may be rendered sensitive include tetracycline (TET), doxycycline (DOX), minocycline, anhydrotetracycline (ATC), and tigecycline.

Also included herein are pharmaceutical compositions comprising the recombinant VACVs described herein. A pharmaceutical composition contains a recombinant VACV and a pharmaceutically acceptable carrier. Exemplary pharmaceutically acceptable carriers include solvents, diluents, dispersion media, lipid carriers and isotonic agents. The compositions optionally comprise antifungal and/or antibacterial agents. The pharmaceutical compositions can be in liquid form, lyophilized form, or aerosol form. In addition, the compositions may include adjuvants to augment the immune response.

The pharmaceutical compositions are used to vaccinate a host such as a mammalian host, such as a human host. The pharmaceutical compositions preferably can be used to stimulate an immune response in the host.

“Nucleic acid sequence” refers to a polymeric form of nucleotides at least 5 bases in length. The nucleotides can be ribonucleotides, deoxyribonucleotides, or modified forms of either nucleotide.

“Operably linked” refers to a juxtaposition wherein the components so described are in a relationship permitting them to function in their intended manner. An expression control sequence operably linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operably linked. Expression control sequences are operatively linked to a nucleic acid sequence when the expression control sequences control and regulate the transcription and, as appropriate, translation of the nucleic acid sequence. Thus, expression control sequences can include appropriate promoters, transcription terminators, a start codon (i.e., ATG) in front of a protein-encoding gene, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant minimal sequence sufficient to direct VACV transcription.

### EXAMPLES

### Example 1

### Testing of Essential VACV Genes Placed Under Control of Natural Tet Operon Elements

Inducible recombinant VACVs were designed by incorporating tetO₂ sequences into the promoters for selected putative essential VACV late genes (A3L, A6L, A7L, D6R, E8R, and F17R) and inserting the tetR gene (under a constitutive promoter) into the VACV genome. The resulting recombinant VACVs were expected to only be able to replicate in the presence of TCs (FIG. 1). Six recombinant VACVs were designed:
1. viA3L (for VACV Inducible A3L) expressing TetR constitutively and having the A3L gene under the control of the tet-responsive A3L promoter (P_{iA3L}).
2. viA6L (for VACV Inducible A6L) expressing TetR constitutively and having the A6L gene under the control of the tet-responsive P₁₁ (F17R) promoter (Pᵢ₁₁).
3. viA7L (for VACV Inducible A7L) expressing TetR constitutively and having the A7L gene under the control of the tet-responsive P₁₁ (F17R) promoter (Pᵢ₁₁).
4. viD6R (for VACV Inducible D6R) expressing TetR constitutively and having the D6R gene under the control of the tet-responsive D6R promoter (P_{iD6R}).
5. viE8R (for VACV Inducible E8R) expressing TetR constitutively and having the E8R gene under the control of the tet-responsive E8R promoter (P_{iE8R}).
6. viF17R (for VACV Inducible F17R) expressing TetR constitutively and having the F17R gene under the control of the tet-responsive F17R promoter (Pᵢ₁₁).

Each virus contains 1) the tetR gene under a constitutive synthetic early/late VACV promoter (P_{E/L}), 2) the selectable E. coli xanthine-guanine phosphoribosyl transferase (gpt) gene and the screening marker enhanced green fluorescent protein (EGFP) gene, as a fusion gene (gpt-EGFP) under control of an additional constitutive synthetic early/late promoter, and 3) a promoter for the essential gene containing operator tetO₂, making it tet-responsive (tet response element) (FIG. 2A).

Late VACV promoter sequences are less conserved than early promoters, but have a very distinct transcriptional initiator sequence. Late VACV promoters commonly consist of an approximately 20 bp long A/T-run, a 6 bp spacer region, and a highly conserved TAAAT(A/G) transcriptional initiator sequence. To make a promoter tet-responsive, the operator sequence is added immediately downstream of the initiator. The P₁₁ promoter of VACV has previously been used as a lac-responsive and tet-responsive promoter. To make the P₁₁ promoter tet-responsive, the tetO₂ sequence (TCCCTATCAGTGATAGAGA; SEQ ID NO. 7) was inserted downstream of the initiator to generate Pᵢ₁₁ (ATATAGTAGAATTTCATTTTGTTTTTTTCTATGCTATAAATATCCCTATCAGT GATAGAGA; SEQ ID NO. 9). To convert the promoters of the chosen putative essential late genes into tet-responsive promoters, their promoters and intergenic regions were carefully studied. The designed promoter sequences are given in Table 1.

TABLE 1
VACV Essential Gene Tet-Responsive Promoter Sequences.

| | | SEQ | |
|---|---|---|---|
| Essential | Inducible | ID | |
| Gene | Promoter | NO. | Promoter sequence |
| A3L | P_{iA3L} | 8 | ATAAGATTGGATATTAAAATCACGCTTTCGAGT |
| | | | AAAAACTACGAATATAAATATCCCTATCAGTGA |
| | | | TAGAGA |
| A6L | Pᵢ₁₁ | 9 | ATATAGTAGAATTTCATTTTGTTTTTTTCTATGC |
| | (based on F17R | | TATAAATATCCCTATCAGTGATAGAGA |
| | promoter, P₁₁) | | |
| A7L | Pᵢ₁₁ | 9 | ATATAGTAGAATTTCATTTTGTTTTTTTCTATGC |
| | (based on F17R | | TATAAATATCCCTATCAGTGATAGAGA |
| | promoter, P₁₁) | | |
| D6R | P_{iD6R} | 10 | ATATATGCTCATATATTTATAGAAGATATCACA |
| | | | TATCTAAATATCCCTATCAGTGATAGAGA |
| E8R | P_{iE8} | 11 | GTATAATCCCATTCTAATACTTTAACCTGATGTA |
| | | | TTAGCATCTTATTAGAATATTAACCTAACTAAA |
| | | | AGACATAACATAAAAACTCATTACATAGTTGAT |
| | | | AAAAAGCGGTAGGATATAAATATCCCTATCAG |
| | | | TGATAGAGA |
| F17R | Pᵢ₁₁(P_{iF17R}) | 9 | ATATAGTAGAATTTCATTTTGTTTTTTTCTATGC |
| | | | TATAAATATCCCTATCAGTGATAGAGA |

The sequences of the tet-responsive promoters for each of the essential genes are shown. In bold are the late transcriptional initiator sequences and underlined are the tetO₂ sequences.

### Materials and Methods:

Generation of Transfer Vectors: A series of cloning steps were used to build the transfer vectors based on existing plasmids and designed synthetic DNA sequences. The final transfer vectors contained: (1) the selectable gpt gene and the screening marker EGFP gene, as a fusion gene (gpt-EGFP) under control of the synthetic early/late promoter P_{E/L}; (2) the repressor gene tetR under the synthetic early/late P_{E/L} promoter; (3) a tet-responsive promoter to direct the expression of the essential gene; and (4) a left border sequence (approximately 600 bp of the gene upstream of the essential gene) and a right border sequence (approximately the first 600 bp of the essential gene or downstream sequences) to serve as recombination sequences for homologous recombination. A schematic of the constructs is shown in FIG. 2A.

The SphI-XmaI fragment (containing the gpt-EGPF gene, tetR gene, and a spacer region) from pCH033 was cloned into the SphI-XmaI site of pCH051 (A3L), pCH052 (A6L), pCH053 (A7L), pCH054 (D6R), pCH055 (E8R), and pCH056 (F17L), which are synthetic plasmids containing the essential gene tet-responsive promoter, and respective left and right recombination sequences (obtained from DNA2.0, Menlo Park, Calif.). This step generated the final transfer vectors: pCH057 (viA3L), pCH058 (viA6L), pCH059 (viA7L), pCH060 (viD6R), pCH061 (viE8R), and pCH062 (viF17R).

Generation of Recombinant Viruses: Homologous recombination was used to precisely insert the genetic elements between the Left and Right Borders of each of the final transfer vectors into the intergenic region between the essential and upstream genes, placing the inducible promoter in front of the essential gene. The recombinant VACVs were generated by standard homologous recombination via transfection of the transfer vectors pCH057, pCH058, pCH059, pCH060, pCH061, or pCH062 into BS-C-1 cell monolayers infected 2 hr earlier at 0.05 plaque-forming units (PFU)/cell with VACV strain Western Reserve (WR) clone 9.2.4.8 (derived from ATCC VR-2035 and obtained from T. Yilma, University of California Davis). Recombinant gpt-positive VACVs were plaque purified on BS-C-1 cells from transfection lysates using gpt selection medium (25 μg/ml mycophenolic acid, 250 μg/ml xanthine, and 15 μg/ml hypoxanthine). All recombinants were plaque-purified in the presence of inducer (1 μg/ml DOX). Expression of EGFP was detected by fluorescence microscopy (Carl Zeiss Axio Observer D1) to ensure that the recombinant viruses were free of parental virus. High-titer stocks were generated by infecting HeLa S3 cells with the recombinant VACVs at a multiplicity of infection (MOI) of 0.1, in the presence of 1 μg/ml DOX. Infected cells were harvested 4 days post-infection by centrifugation at 200×g for 10 min. Cells were then lysed by freezing and thawing, sonicated, and trypsinized. Finally, cell lysates were clarified to remove contaminating cell debris by a second round of sonication and centrifugation at 500×g for 10 min. The overall genomic structure of each recombinant VACV was determined by restriction analysis and PCR analysis of viral DNA (data not shown), which was purified using a small-scale method employing micrococcal nuclease.

### Results:

The ability of the recombinant viruses to grow in the presence or absence of inducer (DOX) was first investigated by plaque assay. Cell monolayers in six-well plates were infected at 40 PFU/well, in the presence of 0, 1, 10, 100, or 1000 ng/ml of DOX; photographs and measurements of isolated VACV plaques were taken 40 hr post-infection with an inverted microscope. For plaque size measurements, cells were stained with crystal violet (0.5% in 20% ethanol), and the diameters of plaques were measured under an inverted microscope (Carl Zeiss Axio Observer D1) with measurement-capable software (AxioVision). Paired t-tests were used to determine the significance of DOX concentration on the plaque size of the different recombinant viruses and WR. Unpaired t-tests were used to determine the difference of plaque sizes between the viruses. All statistical tests were performed with the statistical software Prism (GraphPad Software Inc, San Diego, Calif.). The results are summarized as follows and are also provided in Table 2 and FIG. 3:
The recombinant viA3L is not dependent on DOX for replication.
Recombinant viE8R shows some dependence on DOX for full replication, since plaque size decreased at 1 ng/ml and 0 ng/ml DOX concentration. However, even with no DOX present, viE8R was able to replicate and produce plaques.
Recombinants viA6L, viA7L, viD6R, and viF17R all were unable to form plaques in the absence of DOX, and only singly-infected cells were observed by fluorescence microscopy (data not shown), indicating expression of EGFP but no plaque formation (abortive infections).
Recombinants viD6R and viA7L were able produce plaques at 1000, 100, 10 and 1 ng/ml DOX. At 1 ng/ml DOX plaques were significantly smaller, and no plaques were produced in the absence of DOX (only abortive infections).
The recombinant viA6L produced abortive infections in the absence or presence of 1 ng/ml of DOX, but replicated fully at concentrations of 10 ng/ml or higher.
The recombinant viF17R responded to DOX differently than all other recombinants. The virus appears to be attenuated; plaques were only produced at 1,000 and 100 ng/ml of DOX, and the plaques were much smaller than plaques formed by any of the other viruses at these concentrations. For example, at 100 ng/ml, the average plaque radius was 281.1 μm, thus much smaller than the control (WR) plaques at this concentration (616.9 μm). No plaques were formed at 10, 1 and 0 ng/ml DOX (only abortive infections).

TABLE 2
Average Plaque Radius of Recombinant and Wild-type (WR)
VACVs Grown under Different DOX Concentrations.

| DOX | | | | | | | |
|---|---|---|---|---|---|---|---|
| Concen- | | | | | | | |
| tration | Average Plaque Radius (μm)^{a} | | | | | | |
| (ng/ml) | WR | viA3L | viA6L | viA7L | viD6R | viE8R | viF17R |
| 1000 | 645.4 | 563.3 | 628.2 | 559.9 | 566.4 | 650.4 | 242.7 |
| 100 | 616.9 | 591.9 | 645 | 570.9 | 628.4 | 626.1 | 281.1 |
| 10 | 627.1 | 579 | 593.5 | 590 | 559.5 | 627.3 | AB |
| 1 | 641.5 | 611.6 | AB | 216.2 | 320.1 | 603.8 | AB |
| 0 | 653.1 | 582.4 | AB | AB | AB | 554.2 | AB |

^{a}AB = abortive infection.

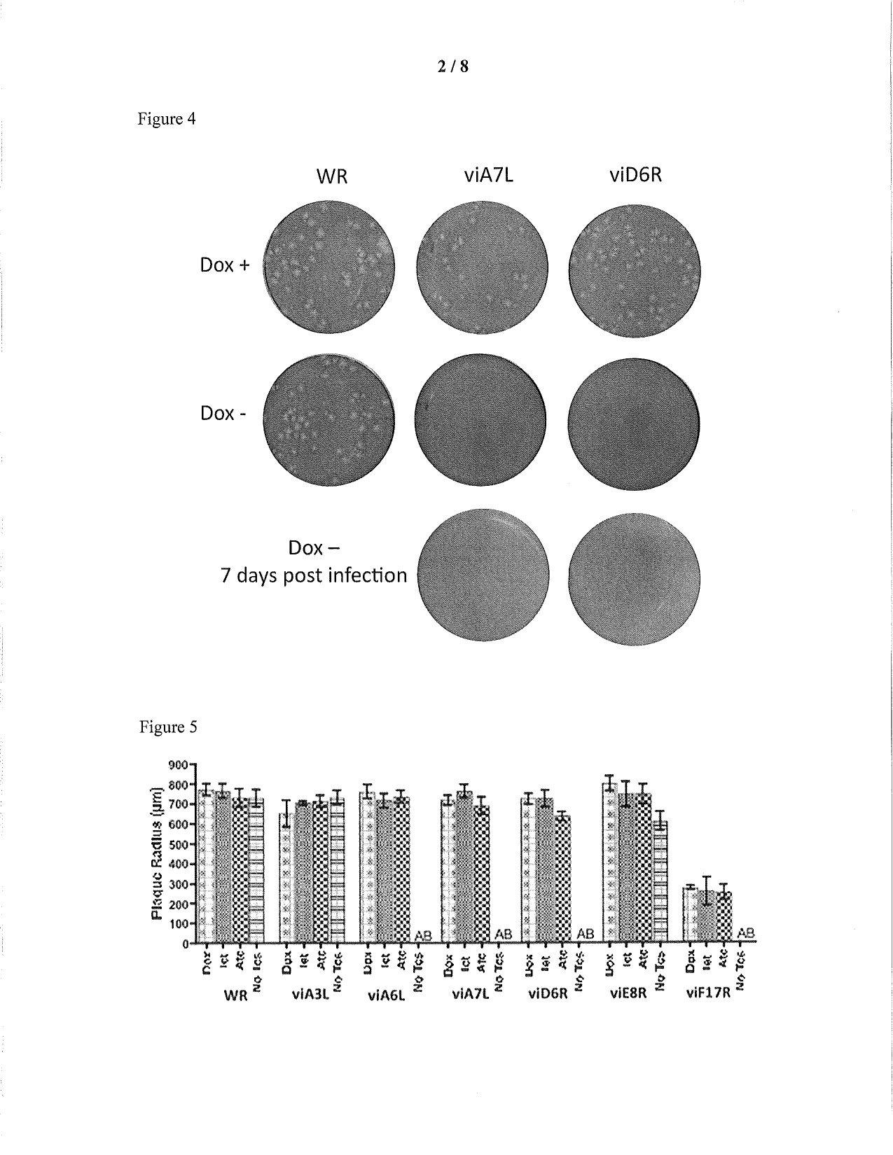

To test the effect of presence or absence of DOX on plaque formation several days after infection, BS-C-1 cell monolayers were infected with wild-type VACV (WR), viA7L, or viD6R in the presence (+) or absence (−) of 1 μg/ml DOX. The cells were stained with crystal violet at either 48 hr post-infection or 7 days post-infection (FIG. 4). The results indicate that recombinant VACVs plaques do not form in the absence of TCs even 7 days after infection.

The ability of the recombinant viruses to grow in the presence of different TCs was investigated by plaque assay. BS-C-1 cell monolayers in 12-well plates were infected at 20 PFU/well, in the presence 1 μg/ml of DOX, TET, or ATC. Photographs and measurements of isolated VACV plaques were taken 40 hr post-infection with an inverted microscope. For plaque size measurements, cells were stained with crystal violet and the diameters of plaques were measured as described above. No significant differences in the size of plaques were observed among the different recombinant viruses and WR grown in the presence of 1 μg/ml DOX, TET, or ATC (FIG. 5), indicating that recombinants viA6L, viA7L, viD6R, and viF17R were all able to form plaques in the presence of different TCs, but not in their absence.

Viral titers at various DOX concentrations were also tested. BS-C-1 cells in 12 well plates were infected at an MOI of 0.01 with WR, viA3L, viA6L, viA7L, viD6R, viE8R, or viF17R. After 1 h, virus was aspirated and medium with 1000, 100, 10, 1, or 0 ng/ml DOX was added. Cells were collected either 0 hr (immediately after the 1 hr infection) or at 48 hr post-infection. The intracellular fraction of virus was collected: cells were removed from the wells, centrifuged at 300×g for 10 min to pellet the cells, supernatant (containing extracellular virus) was removed and the cells were resuspended in 500 μl of medium. The intracellular fraction was processed and titered on BS-C-1 cells as previously described, in the presence of 1 μg/ml of DOX.

With the exception of viD6R, the viral titers (yield) obtained 48 hr after infection of BS-C-1 cells at an MOI of 0.01 in the presence of various DOX concentrations reflected the plaque sizes that were observed in the single plaque analysis (data not shown). As expected, WR, viA3L, and viE8R showed no dependence on DOX for viral replication, while the titers of viA6L, viA7L, and viF17R were dependent on DOX. Recombinant viA6L yielded a high titer for 1000, 100, and 10 ng/ml DOX, which dropped swiftly from 8.5×10⁶ PFU/ml at 10 ng/ml to 40 PFU/ml at 1 ng/ml DOX and remained close to that level at 0 ng/ml DOX. Recombinant viA7L showed a more gradual decrease in titer. The titer dropped from 7.2×10⁶ PFU/ml at 10 ng/ml DOX to 1.9×10⁴ PFU/ml at 1 ng/ml and finally to 0 PFU/ml in the absence of DOX. The results for viA6L and viA7L mimic what was observed when measuring plaque size.

The titers (yield) of viD6R did not follow the observed plaque sizes. When measuring plaque size, no plaques were seen in the absence of DOX, however the titer of viD6R at 0 ng/ml was 1.05×10⁵ PFU/ml, much higher than what was observed for viA7L or viA6L (both of which also did not produce plaques in the absence of DOX). The attenuation of viF17R was also apparent in the titers. Although no viral plaques were observed at 10, 1, or 0 ng/ml DOX, the viral titers at 10 and 1 ng/ml DOX were increased in comparison to the titers in absence of DOX.

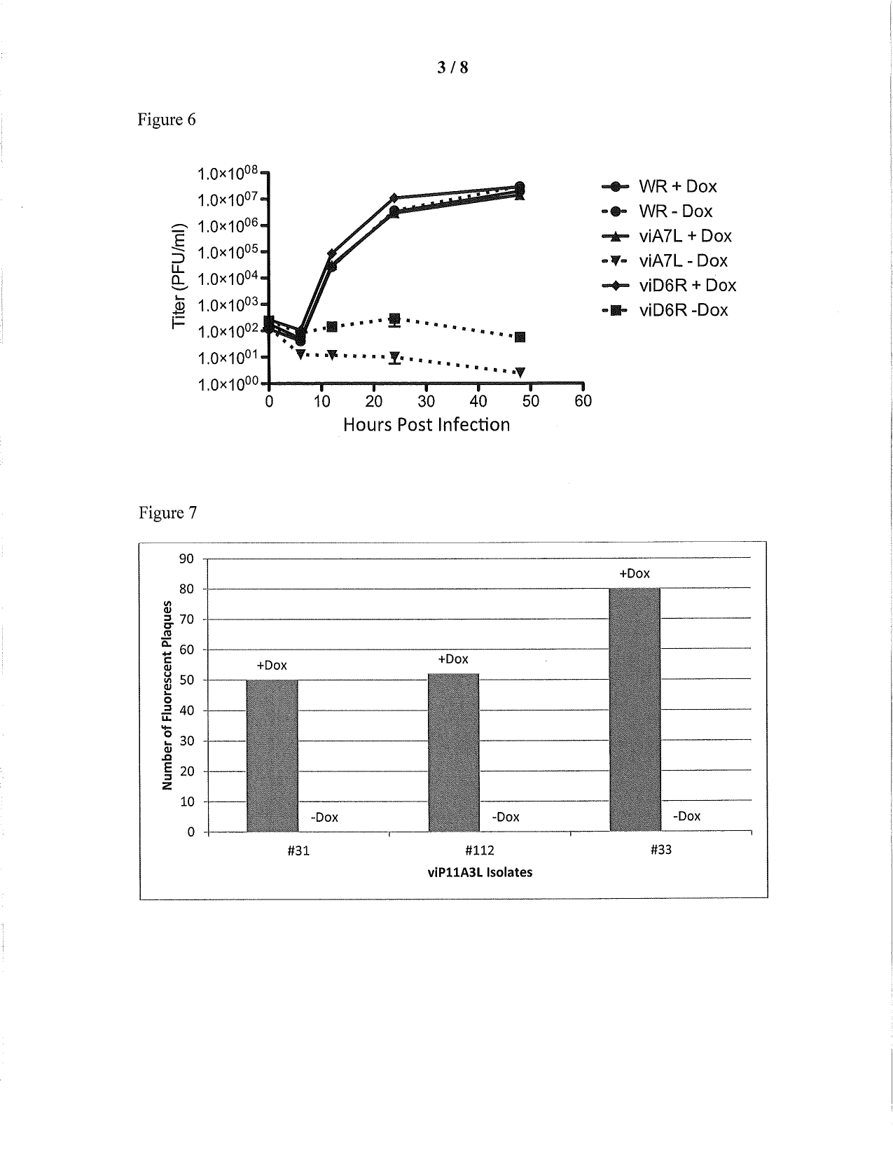

To test the effect of DOX on viral replication at a low MOI, BS-C-1 cell monolayers were infected with 0.01 PFU of WR, viA7L, or viD6R per cell in the presence (+) or absence (−) of 1 μg/ml DOX in triplicates. The cells were collected at the indicated times after infection and viral titers were determined by plaque assay in the presence of 1 μg/ml DOX (FIG. 6). The results clearly indicate that the recombinant VACVs are able to replicate to the same levels as WR (wild-type) VACV in the presence of DOX, and unable to replicate in its absence.

### Discussion:

Without being held to theory, it is believed that the ability of viA6L to replicate at relatively low DOX concentrations (10 ng/ml) at a rate similar to wild-type would allow the antibiotic dose given with the vaccine to be kept at a low and safe level for the vaccine recipient. The abrupt cease of viral replication between 10 ng/ml and 1 ng/ml that viA6L displays may also be a desirable trait in a DOX dependent vaccine and therapeutic vectors. If the vaccine can be induced with a low dose of DOX, when treatment is stopped the DOX concentration within the body should fall to the critical concentration quickly, rapidly stopping virus replication. Similarly, viA7L is also a good vector candidate; however, this virus did replicate at reduced levels at 1 ng/ml DOX. For replication to be induced at wild-type levels, 10 ng/ml DOX was required. Without being held to theory, this suggests that for a good take to occur upon vaccination, viA7L may require a dose of DOX similar to A6L. However, viral replication could be more difficult to stop, as 1 ng/ml DOX would be sufficient to induce the replication of viA7L. It is hypothesized that if an adverse reaction occurs after vaccination once DOX treatment is stopped, the level within the body would have to decrease to below 1 ng/ml (rather than to below 10 ng/ml for viA6L) to stop viral replication and the adverse event.

The recombinant viD6R appears to be a good vector candidate based on the plaque size assays, which show no evidence of viral replication in the absence of DOX. However, the viral titers of viD6R do increase even in the absence of DOX.

While viF17R was inducible by TCs, it appears to be attenuated which is not ideal for smallpox vaccination. This recombinant was unable to replicate at levels similar to wild-type even in the presence of high levels of inducer. However, it did form abortive infections even at 10 ng/ml of DOX, which may make it a suitable vector for applications where an attenuated vector is desirable or sufficient. Recombinant viA3L and viE8R were not dependent on TCs for replication, and in their current state are not good vaccine vector candidates. However, this does not indicate that these genes may not be useful for controlling VACV replication, since there is strong evidence suggesting that both viA3L and viE8R are essential for VACV replication.

### Example 2

### Inducible Recombinant VACVs Utilizing Natural Tet Operon Elements to Control the Expression of the A3L and E8R Genes Under the Inducible P₁₁ Promoter

Inducible recombinants viA6L, viA7L, viD6R, and viF17R all displayed dependence on the presence of inducer, while viA3L and viE8R did not. As there is evidence in the literature that both A3L and E8R genes are critical for viral replication, this result was unexpected. Since both viA3L and viE8R were designed utilizing their natural promoters, it is possible that the tetO₂ sequence was placed in a location that leads to inadequate repression of the essential gene in the absence of inducer. Replacing the natural promoter regions with the inducible P₁₁ promoter, which is tet-responsive, could allow for the generation of inducible recombinant VACVs for the A3L and E8R genes.

### Materials and Methods:

Generation of Transfer Vectors: New transfer plasmids were designed from existing transfer plasmids. Plasmids contained a number of genetic elements, including: (1) the gpt gene for selection and EGFP gene for screening, as a gpt-EGFP fusion gene under the control of the VACV constitutive synthetic early/late promoter (P_{E/L}); (2) tetR, the repressor gene, under the control of an additional constitutive synthetic P_{E/L} promoter; and (3) the essential gene (A3L or E8R) under the control of the inducible P₁₁ promoter containing a tetO₂ sequence immediately after the transcriptional initiator sequence (TAAATA). To generate a transfer vector placing the A3L gene under the control of the inducible P₁₁ promoter, restriction enzyme digestion was utilized to excise the P₁₁ promoter from pCH059 and the natural A3L promoter from pCH057. The NcoI-EagI fragment from pCH059 containing EGFP and the P₁₁ promoter was cloned into the NcoI-EagI site of pCH057, generating the new transfer plasmid pBJ107. A similar procedure was utilized to generate a plasmid placing the E8R gene under the control of the inducible Pᵤ promoter. The NheI-EagI fragment from pCH059 containing gpt-EGFP and the P₁₁ promoter was cloned into the NheI-EagI site of pCH061, generating the new transfer plasmid pBJ108.

Generation of Recombinant Viruses: The recombinant VACVs were generated by transfection of BS-C-1 cells infected with VACV (WR strain) at 0.05 PFU/cell with plasmids pBJ107 or pBJ108, as described in Example 1. The recombinant VACVs were named viP11A3L (derived from pBJ107) and viP11E8R (derived from pBJ108) and were plaque purified as described in Example 1. Plaque isolates used for preliminary testing underwent two or more rounds of plaque purification at the time of testing.

### Results:

The recombinant VACVs were tested as described in Example 1. All isolates for viP11A3L formed fluorescent plaques in the presence of DOX (FIG. 7). However, in the absence of DOX, no plaques were observed and only single EGPF⁺ cells (abortive infections) could be detected by fluorescence microscopy. No evidence of wild-type virus (non-fluorescent plaques) was observed for any of the isolates.

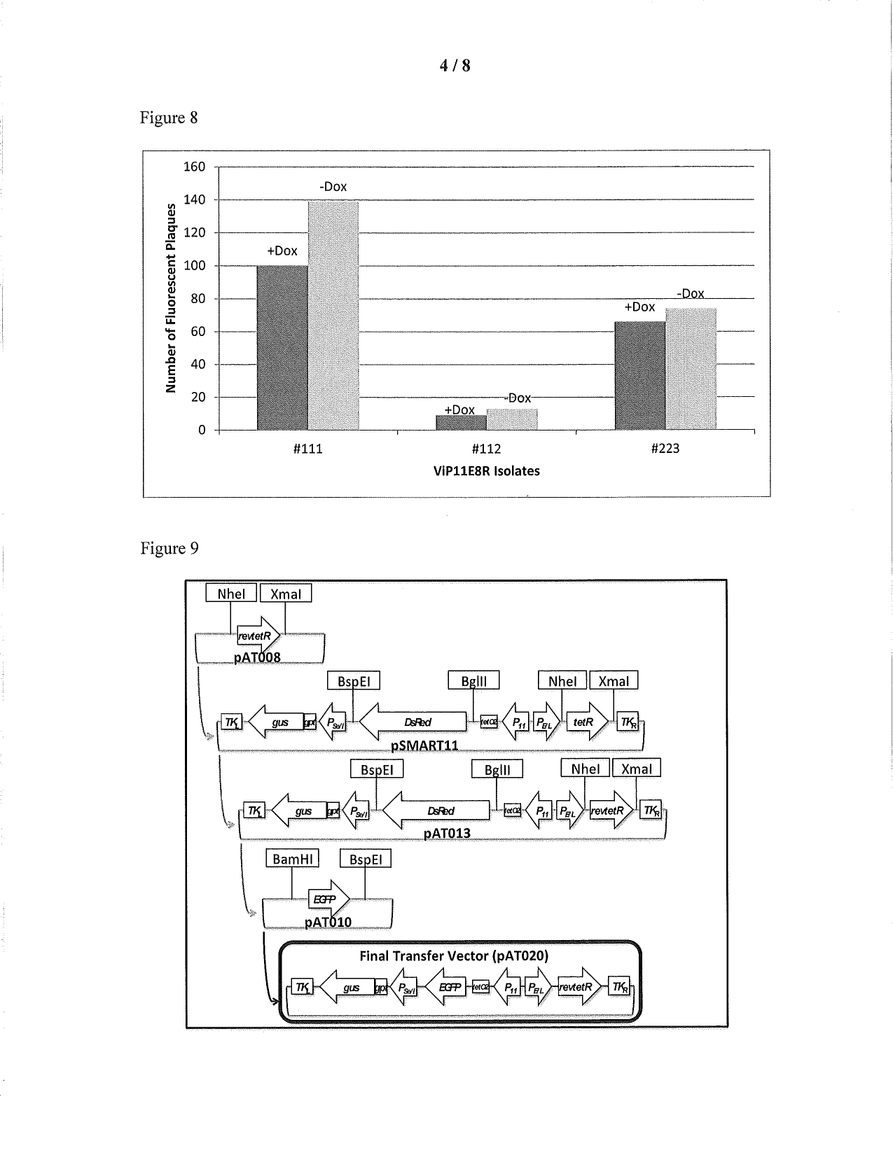

All plaque isolates for viP11E8R showed little difference between the number of fluorescent plaques in the presence and absence of DOX (FIG. 8). Isolate #111 formed 100 fluorescent plaques in the presence of DOX and 139 fluorescent plaques in the absence of DOX. Isolate #112 formed 9 fluorescent plaques in the presence of DOX and 13 in the absence of DOX. Isolate #223 formed 66 fluorescent plaques in the presence of DOX and 74 in the absence of DOX. However, the plaques produced in the absence of DOX tended to be smaller than the plaques produced in the presence of DOX (data not shown).

### Discussion:

The recombinant viP11A3L exhibited dependence on DOX for viral replication. In the absence of DOX, no fluorescent plaques were observed, indicating the repressor protein TetR was bound to the operator sequence tetO₂ located in the P₁₁ promoter, preventing transcription of the A3L gene. This also indicates that the A3 protein is indeed essential for VACV replication. In contrast, viP11E8R was able to replicate in the presence or absence of DOX; similar numbers of fluorescent plaques were observed in both conditions, albeit plaques in the absence of DOX tended to be smaller. The observed results indicate viP11E8R lacks dependence on DOX for viral growth and provide evidence that the E8 protein may not be essential for VACV replication. Without being held to theory it is believed that leaky transcription of the E8R gene from the strong P₁₁ promoter may be sufficient to permit viral replication in the absence of DOX, and therefore the inventors hypothesize that the use of a weaker promoter may be required to generate an inducible VACV based on the E8R gene.

### Example 3

### Development of a Repressible VACV Expression System Assembled with Reverse Tet Repressor Mutants and Other Elements from the Tet Operon

The goal of this study was to develop and test a repressible system that functions in VACV. Elements from the tet operon of E. coli and reverse mutants of the tetR (revtetR) gene were used to generate recombinant VACVs that repress the expression of the EGFP reporter gene in the presence of TCs.

### Materials and Methods:

Generation of Transfer Vectors: The construction of the vRG0 transfer vector is detailed in FIG. 9. Briefly, the NheI and XmaI fragment of pAT008, a designed synthetic plasmid (DNA2.0) that contains the revtetR (L17G) gene, was subcloned into the same sites of pSMART11. The resulting plasmid (pAT013) contains the revtetR gene under the control of the synthetic early/late VACV P_{E/L} promoter, the DsRed gene under the control of the natural late VACV P₁₁ promoter (back-to-back with the P_{E/L}) with a 19 bp tetO₂ placed directly downstream from P₁₁ and upstream from the transcriptional start site, and the gpt-gus fusion gene under the control of the synthetic VACV P_{Sel} promoter, flanked by segments of the VACV thymidine kinase (TK) gene (left, TK_{L} and right, TK_{R}), which allows for homologous recombination with the VACV TK genomic region. The final transfer vector pAT020 was generated by transferring the EGFP gene from pAT010 into the BglII and BspEI sites of pAT013. A control transfer vector (pAT018) for the generation of a recombinant VACV constitutively expressing EGFP under the P₁₁ promoter (vRGc) was generated by transferring the revtetR gene into plasmid pSMART10, and by replacing the DsRed gene with the EGFP gene as described above. Additionally, transfer vectors with different mutants of the revtetR gene (Table 3) were generated by subcloning the mutant revtetR synthetic genes into the NheI and XmaI sites in pAT020.

TABLE 3
Reverse TetR (RevTetR) Mutants Tested.

| Recombinant | | |
|---|---|---|
| VACV | revtetR Gene | Mutations |
| vRG0 | L17G | L17G |
| vRG1 | Original rtTA | E71K, D95N, L101S, G102D |
| vRG2 | Advanced rtTA | S12G, E19G, A56P, A148E, H179R |
| | (rtTA-M2) | |
| vRG3 | rtTA-S2 | E19G, A56P, A148E, H179R |
| vRG4 | rtTA-M1 | S12G, E19G, A56P |
| vRG5 | rtTA-19-56R | E19G, A56P |

Generation of Recombinant Viruses: The recombinant VACVs vRG0, vRG1, vRG2, vRG3, vRG4, vRG5 (Table 3), and vRGc (a positive control virus without tetO₂, thus constitutively expressing EGFP under the P₁₁ promoter) were generated via standard homologous recombination by transfection of the transfer vectors into BS-C-1 cell monolayers infected with VACV strain WR (also used as the negative control) at a MOI of 0.05 PFU/cell. Recombinant VACVs were plaque purified in gpt selection medium; plaques were visualized either with substrate 5-bromo-4-chloro-3-indolyl-β-D-glucuronide (X-Gluc) or by selecting and collecting only EGFP⁺ expressing plaques detected via fluorescence microscopy. The purity of the recombinant VACVs (absence of parental virus) was confirmed by checking multiple dilutions of stocks in selection-free media for the presence of non-fluorescent (EGFP⁻) plaques. High titer stocks of each recombinant VACV were generated as described in Example 1, in the absence of TCs.

Determination of EGFP Expression: BS-C-1 cell monolayers in multiwell culture plates were infected with the VACVs (30 PFU or an MOI of 1) in the presence of medium only or various concentrations of ATC (with or without various concentrations of MgCl₂, allowed to form a complex for 1 hr before use), TET, or DOX. At 2 days post-infection, EGFP expression by isolated plaques or infected cells were determined by quantitative fluorescence microscopy (as described below). In certain instances, cell monolayers were washed with 1× phosphate-buffered saline (PBS) pH 7.2, resuspended in equal parts of 1×PBS and 2% neutral buffered formalin (1% final concentration), and analyzed with a fluorescence plate reader (as described below) within 24 hr.

Imaging and Image Quantification: Infected cells and plaques were imaged using an inverted fluorescence microscope (Axio Observer D1, Carl Zeiss, Thornwood, N.Y.) with or without a green bandpass filter (XF100-2, Omega Optical, Brattleboro, Vt.). EGFP expression levels were quantified with the AxioVision software, release 4.8.1 (Carl Zeiss). Quantification of fluorescence of infected cells were also performed with a fluorescence plate reader (Synergy HT Multi-Mode Microplate Reader, BioTek Instruments, Winooski, Vt.) using a 485/20 nm excitation and a 528/20 nm emission filter pair. EGFP expression levels are quantified with the KC4 v3.4 software (BioTek Instruments).

### Results:

vRG0 Expresses EGFP at High Levels in the Absence of TCs: The first mutant revtetR tested in this system was based on a TetR mutant with a single amino acid change at position 17 that changes leucine to glycine (L17G). Although this L17G RevTetR mutant was characterized in bacteria (E. coli), it was appealing to test initially in this system, since a single mutation is less likely to alter other important regions on the repressor protein. In addition, this mutant was characterized as a single domain protein, unlike most other RevTetR mutants that were characterized as fusion proteins with the VP16 transcriptional activator from herpes simplex virus. Moreover, it is important to note that ATC (a TC-derivative) was used to characterize the L17G mutant in E. coli.

In the absence of TCs, BS-C-1 cells infected with vRG0, a positive control virus (vRGc), or a negative control virus (WR) displayed the expected cytopathic effect 2 days post-infection when observed under brightfield microscopy. Additionally, vRG0 and vRGc plaques displayed similar high levels of EGFP expression under fluorescence microscopy, while WR-infected cells did not (data not shown).

EGFP Expression Levels by vRG0 Were not Repressed Even in the Presence of High Concentrations of TCs: To test whether vRG0 was repressible by TCs, BS-C-1 cells were infected with vRG0 in the presence of increasing concentrations of ATC, TET, or DOX. The cytopathic effect observed by brightfield microscopy 2 days post-infection was indistinguishable from the cytopathic effect observed from cells treated with ATC and 10 μg/ml or less of TET or DOX (data not shown). However, cells infected in the presence of 100 μg/ml of TET or DOX exhibited morphology associated with cell toxicity, and as a result the plaques were smaller in comparison to infected cells treated with lower concentrations of TET or DOX. Cells infected in the presence of ATC did not incur any toxicity because lower concentrations were used in comparison to the TET and DOX, as ATC has the highest affinity for RevTetR. Increasing concentrations of TCs did not decrease EGFP expression in vRG0 plaques when observed by fluorescence microscopy. In the presence of 100 μg/ml of TET or DOX, infected cells exhibited lower EGFP expression, likely due to cell toxicity.

Since TCs complexed to MgCl₂ bind to the TetR repressor with higher affinity than TCs alone (a commonly used strategy when studying induced conformation of the repressor by x-ray crystallography), ATC was incubated with MgCl₂ to allow for complex formation. Cells infected with vRG0 in the presence of various concentrations of ATC-MgCl₂ displayed typical cytopathic effect 2 days post-infection at 20 mM or below of MgCl₂, while at the highest concentration of MgCl₂ (100 mM MgCl₂), the cells exhibited toxicity-associated morphology (data not shown). When plaques were observed by fluorescence microscopy, there was no EGFP repression in the presence of increasing concentrations of the ATC-MgCl₂ complex (data not shown). In addition, at the highest concentrations of ATC-MgCl₂ complex tested (100 mM), no fluorescent plaques were observed, likely as a result of MgCl₂ cell toxicity. Thus, it can be concluded that that vRG0 was not repressible and decided to test new revtetR mutants in Table 3.

RevTetR Mutants Lead to Different EGFP Expression Levels in the Absence of TCs: Five new recombinant VACVs (vRG1, vRG2, vRG3, vRG4, and vRG5) were generated (Table 3), each expressing a different mutant tetR gene previously shown to display the reverse phenotype in eukaryotic systems when fused to a VP 16 domain from herpes simplex virus that induces gene expression. Since the VP16 transactivator would not function in VACV, as VACV relies on its own specialized cytoplasmic transcription machinery, the RevTetR mutants were tested without the VP16 transactivator. Additionally, these mutants were initially characterized with the TC derivative DOX.

In the absence of TCs, BS-C-1 cells infected with the VACVs displayed the expected cytopathic effect 2 days post-infection under brightfield microscopy. When observed under fluorescence microscopy, vRG5, vRG0, and vRGc plaques displayed similar high levels of EGFP expression (data not shown). The other recombinants had varying lower levels of expression, with vRG3 and vRG4 having equal levels of EGFP expression, vRG2 less, and vRG1 least.

RevTetR Mutants Lead to Different Repression of EGFP Expression Levels in the Presence of TCs: BS-C-1 cells were infected with the different VACVs at an MOI of 11n the absence or presence of various concentrations of TCs. Two days post-infection cells were imaged by brightfield and fluorescence microscopy and EGFP expression was subsequently quantified. Based on fluorescence microscopy images and quantification (data not shown), all RevTetR mutants displayed varying levels of EGFP repression, generally being most sensitive to DOX, followed by ATC and then TET. vRG0 was repressible, but EGFP expression remained high even at the highest concentrations of TCs tested. In the presence of DOX, only vRG1 and vRG2 fully repressed EGFP expression to the levels of the negative control WR at 0.1 μg/ml, although EGFP expression levels by vRG1 in the absence of TCs were the lowest (data not shown). In addition, vRG4 was able to be fully repressed at 1 μg/ml of DOX, while vRG3 was fully repressed at 10 μg/ml of DOX. In the absence of TCs, vRG5 was able to express EGFP at the same level as vRGc, although it was not able to become fully repressed based on fluorescence imaging quantification.

To potentially increase the sensitivity and reproducibility of the fluorescence quantification and to better ascertain the repressible systems that exhibit full repression, a fluorescence plate reader (fluorometer) was employed. BS-C-1 cells were infected with the VACVs at an MOI of 1 in multiwell plates in the absence or presence of increasing concentrations of TCs, and 2 days post-infection cells were fixed and read on the multiwell fluorescence reader (FIGS. 10 (ATC), 11 (TET) and 12 (DOX)). The results follow essentially the same trends observed by fluorescence imaging. In addition, it seems that lower levels of EGFP expression were detected with increased sensitivity, although there was more variance at higher levels of EGFP expression. Thus, for the purposes of determining EGFP repression by the recombinant VACVs, the fluorometer seems more appropriate as it is more sensitive at lower levels of expression.

Full Repression of EGFP Expression is Achievable in VACV: To determine the TC concentration that fully represses EGFP expression, BS-C-1 cells were infected with the VACVs in the absence or presence of increasing concentrations of TCs, and 2 days post-infection cells were fixed and read on the fluorescence multiwell reader. In the presence of increasing concentrations of ATC (0.1 ng/ml-1,000 ng/ml), vRG1-vRG5 showed a repressible dose response (FIG. 13). vRG1 was fully repressed to the levels of the negative control WR in the presence of 100 ng/ml ATC, while vRG2, vRG3, and vRG4 were fully repressed in the presence of 1,000 ng/ml ATC. Only vRG5 and vRG0 were not fully repressed at 1,000 ng/ml of ATC.

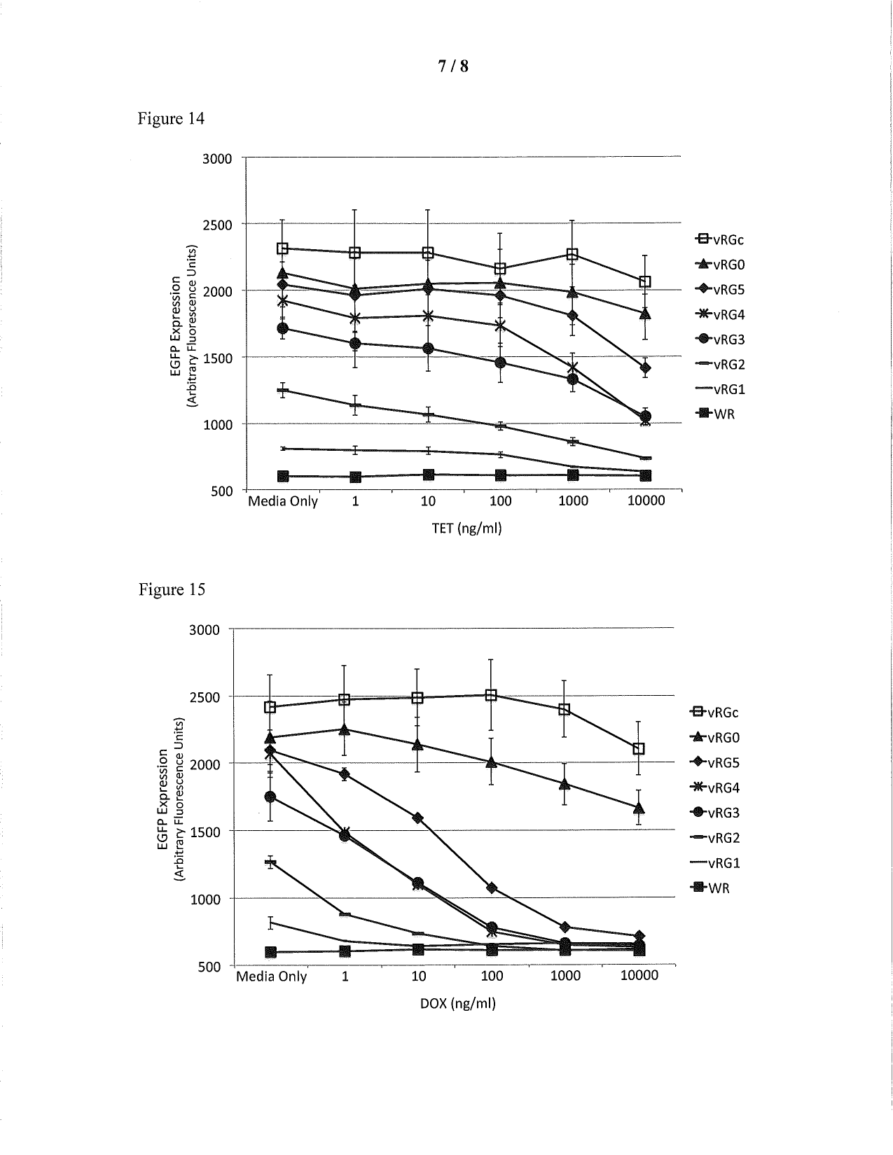

In the presence of increasing concentrations of TET (1 ng/ml-10,000 ng/ml), the expression in vRG1-vRG5 also showed a repressible dose response (FIG. 14). However, vRG1 was the only recombinant that became fully repressed to the levels of WR. In the presence of increasing concentrations of DOX (1 ng/ml-10,000 ng/ml), all recombinant VACVs show a repressible dose response (FIG. 15). With the exception of vRG0 and vRG5, all other recombinants were fully repressed by concentrations of DOX within the range tested; however, some recombinant VACVs are more sensitive to DOX than others. vRG1 was fully repressed at 10 ng/ml of DOX, vRG2 was next at 100 ng/ml, and vRG3 and vRG4 were next at 1,000 ng/ml. At the highest DOX concentration tested (10,000 ng/ml), vRG5 seems to be mostly, but not completely repressed.

### Discussion:

All of the RevTetR repressor mutants tested in this VACV expression system displayed a repressible phenotype in the presence of TCs, albeit with unique phenotypes. For example, vRG0 (with the single L17G tetR mutant) was only marginally repressible, while the recombinant VACV most sensitive to TCs (vRG1) was also the one with the lowest level of EGFP expression in the absence of TCs. As a general trend, the higher the level of EGFP expression in the absence of TCs, the higher the TC concentration needed to completely repress the system.

### Example 4

### Repressible Recombinant VACVs Controlling the Essential Gene A6L

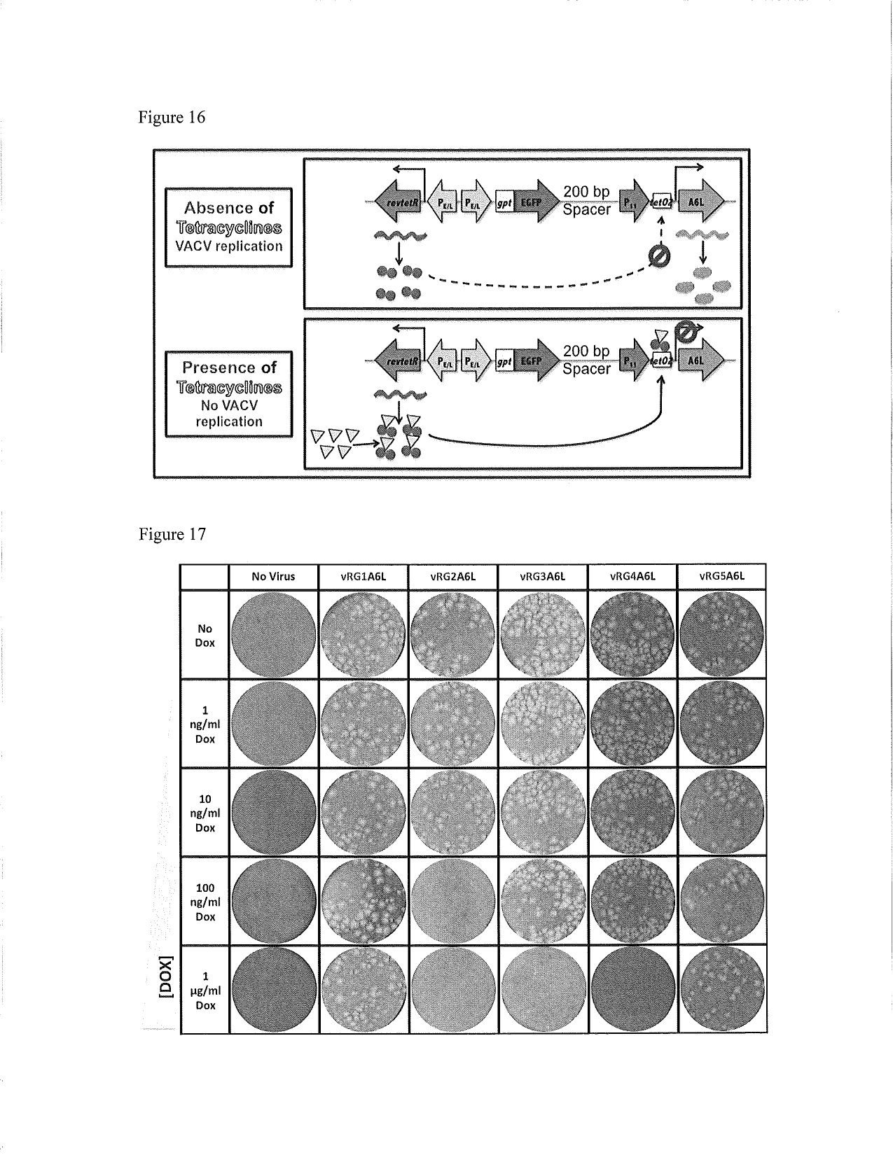

In this study, elements from the tet operon of E. coli and reverse mutants of the tetR (revtetR) gene were used to generate recombinant VACVs that repress the expression of the VACV gene A6L, which was shown to be essential for virus replication in Example 1. In the absence of TCs, RevTetR (produced constitutively) does not bind to the tetO₂ operator and does not block A6L expression, resulting in virus replication. In the presence of TCs, RevTetR undergoes a conformational change and binds to tetO₂, blocking transcription of A6L and consequently, viral replication (FIG. 16).

### Materials and Methods:

Generation of Transfer Vectors: The construction of the pCH067, pCH068, pCH069, pCH070, and pCH071 transfer vectors are detailed briefly. The NheI and XmaI fragments of pAT035, pAT036, pAT037, pAT038, and pAT039, containing the various mutants of the revtetR gene, were subcloned into the same sites of pCH058. The resulting transfer vector plasmids contain the revtetR gene under the control of the synthetic constitutive early/late VACV P_{E/L} promoter, the gpt-EGFP fusion gene under the control of the synthetic VACV P_{Sel} promoter, a 200 bp spacer, the A6L gene under the control of the natural late VACV P₁₁ promoter with a 19 bp tetO₂ placed directly downstream from P₁₁ and upstream from the transcriptional start site (FIG. 2B).

Generation of Recombinant Viruses: The recombinant VACVs vRG1A6L, vRG2A6L, vRG3A6L, vRG4A6L, and vRG5A6L (Table 4) were generated via standard homologous recombination by transfection of the transfer vectors into BS-C-1 cell monolayers infected with VACV strain WR as described in the previous examples, always in the absence of TCs.

TABLE 4
RevTetR Mutants Used to Generate the Replication
Repressible A6L VACV Recombinants.

| Recombinant | | |
|---|---|---|
| VACV | revtetR Gene | Mutations |
| vRG1A6L | Original rtTA | E71K, D95N, L101S, G102D |
| vRG2A6L | Advanced rtTA | S12G, E19G, A56P, D148E, H179R |
| | (rtTA-M2) | |
| vRG3A6L | rtTA-S2 | E19G, A56P, D148E, H179R |
| vRG4A6L | rtTA-M1 | S12G, E19G, A56P |
| vRG5A6L | rtTA-19/56R | E19G, A56P |

### Preliminary Results:

To determine if replication of these recombinant viruses can be repressed in the presence of TCs, BS-C-1 cells were infected with vRG1A6L-vRG5A6L (80-100 PFU/well) in the absence or presence of increasing concentrations of DOX and 2 days post-infection, infected cells were imaged using microscopy or stained with crystal violet and the plaques were imaged. In the absence of DOX, all recombinants displayed the expected cytopathic effect 2 days post-infection (FIG. 17). In the presence of increasing concentrations of DOX, BS-C-1 cells infected with only vRG2A6L, vRG3A6L, and vRG4A6L were repressed by DOX as no cytopathic effect (plaque formation) was observed. Interestingly, vRG2A6L in the presence of 100 ng/ml DOX and vRG3A6L in the presence of 1 μg/ml of DOX had almost full repression of viral replication, producing only a few small plaques. More importantly, viral replication was fully repressed as no cytopathic effect was observed for vRG3A6L and vRG5A6L in the presence of 1 μg/ml of DOX.

Abortive Infections were Observed in vRG2A6L, vRG3A6L, and vRG4A6L in the Presence of DOX: An abortive infection is defined as a virus that is able to gain entry into the cell, but the no infectious progeny is produced and hence no cytopathic effect. To determine if the VACV recombinants produce abortive infections, BS-C-1 cells were infected with all the VACV recombinants (80-100 PFU/well) in the presence of 5 μg/ml DOX. The cells were observed 2 days post-infection using fluorescence microscopy. While vRG1A6L and vRG5A6L displayed fluorescent plaques (viral replication was not repressed), vRG2A6L, vRG3A6L and vRG4A6L exhibited no cytopathic effect and only produced single fluorescent cells distributed randomly (vRG2A6L) or in small clusters (vRG3A6L and vRG4A6L) (data not shown). Thus, vRG2A6L, vRG3A6L, and vRG4A6L seem to produce abortive infections in the presence of DOX, with vRG2A6L only producing single fluorescent plaques.

### Discussion:

In the presence of increasing concentrations of DOX, vRG1A6L and vRG5A6L were not DOX-responsive. In the case of the fluorescent cell clusters observed for vRG3A6L and vRG4A6L, there is a possibility that these recombinant virus isolates were not fully purified, and any wild-type (WR) still present could be complementing the growth of the transcriptionally repressed A6L, and therefore these results should be considered preliminary. Additional plaque purifications to obtain completely purified virus and more elaborate testing will be performed to fully characterize these conditionally repressible VACV recombinants.

### LIST OF IMPORTANT ABBREVIATIONS USED

VACV=vaccinia virus
tet=tetracycline (as in tet operon)
TC=tetracycline as drug class
TET=tetracycline antibiotic
DOX=doxycycline
ATC=anhydrotetracycline
MOI=multiplicity of infection
PFU=plaque-forming unit
SEM=standard error of the mean
gpt=xanthine-guanine phosphoribosyl transferase
EGFP=enhanced green fluorescent protein
PBS=phosphate-buffered saline
TK=Thymidine kinase

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A recombinant vaccinia virus comprising:
a first recombinant nucleic acid comprising a tet response element and a nucleic acid encoding a conditional replication gene product, wherein the tet response element is operably linked to the nucleic acid encoding the conditional replication gene product; and
a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor, and wherein the second recombinant nucleic acid is located in a non-essential region of the vaccinia virus genome or in an intergenic region of the vaccinia virus genome; and
wherein the conditional replication gene product is a vaccinia virus gene product essential for virus replication, wherein the essential vaccinia virus gene product is not the A14 protein, and
wherein expression of the conditional replication gene product is inducible or repressible by a tetracycline antibiotic.

2. The recombinant vaccinia virus of claim 1, wherein the intergenic or non-essential region of the vaccinia virus genome is immediately upstream from the conditional replication gene.

3. The recombinant vaccinia virus of claim 1, wherein the tet response element comprises a vaccinia virus promoter operably linked to a tet operator.

4. The recombinant vaccinia virus of claim 1, wherein the tet response element comprises two or more tet operators.

5. The recombinant vaccinia virus of claim 1, wherein the recombinant vaccinia virus is replication competent either in the presence or absence of tetracycline antibiotics.

6. The recombinant vaccinia virus of claim 1, wherein the second recombinant nucleic acid encodes a tet repressor.

7. The recombinant vaccinia virus of claim 1, wherein the second recombinant nucleic acid encodes a reverse tet repressor.

8. The recombinant vaccinia virus of claim 1, wherein the tet response element is inserted between a transcriptional initiator of the conditional replication gene and a translational start site of the conditional replication gene.

9. The recombinant vaccinia virus of claim 8, wherein the second recombinant nucleic acid comprising an expression control sequence and a nucleic acid encoding a tet repressor or a reverse tet repressor is inserted into the viral genome in an intergenic region between the nucleic acid encoding the conditional replication gene product and its upstream gene.

10. The recombinant vaccinia virus of claim 1, wherein the tet response element overlaps the transcriptional initiator of the conditional replication gene.

11. The recombinant vaccinia virus of claim 1, wherein the second nucleic acid is inserted into the viral genome in a way that does not substantially affect the viability of the virus.

12. The recombinant vaccinia virus of claim 1, wherein the essential vaccinia virus gene product is the A6L gene product, the A7L gene product, the D6R gene product, the F17R gene product, or the A3L gene product.

13. The recombinant vaccinia virus of claim 1, wherein a conditional replication gene product is expressed or repressed in the presence of a tetracycline antibiotic.

14. The recombinant vaccinia virus of claim 13, wherein the tetracycline antibiotic is selected from tetracycline, doxycycline, minocycline, anhydrotetracycline and tigecycline.

15. A method of vaccinating an individual against smallpox, comprising
administering to the individual the vaccinia virus of claim 1 in an amount sufficient to elicit an immune response.

16. The method of claim 15, further comprising administering a tetracycline antibiotic in an amount sufficient to induce or repress the production of the conditional replication gene product, wherein induction provides vaccinia virus replication, and wherein repression reduces or ceases vaccinia virus replication.

17. The recombinant vaccinia virus of claim 1, wherein the recombinant vaccinia virus further comprises an exogenous gene operably linked to an expression control sequence for the exogenous gene, wherein the product of the exogenous gene is a therapeutic protein.

18. The recombinant vaccinia virus of claim 17, wherein the therapeutic protein is a foreign antigen for a human or an animal pathogen, a tumor antigen, an immunostimulatory molecule, a gene that enhances the oncolytic potential of the recombinant vaccinia virus, or a gene used for tumor imaging or vector tracking in vivo.

19. The recombinant vaccinia virus of claim 1, wherein the first recombinant nucleic acid comprises an exogenous gene operably linked to the tet response element, wherein the product of the exogenous gene is a gene product that is toxic to a mammalian host or cells, lowers the fitness of the recombinant vaccinia virus, or interferes with the replication of VACV.

20. A recombinant vaccinia virus comprising:
a first recombinant nucleic acid comprising a tet response element and a nucleic acid encoding a conditional replication gene product, wherein the tet response element is operably linked to the nucleic acid encoding the conditional replication gene product; and
a second recombinant nucleic acid comprising an expression control sequence and a nucleic acid encoding a tet repressor or a reverse tet repressor that conditionally binds the tet response element, wherein the expression control sequence is operably linked to the nucleic acid encoding the tet repressor or a reverse tet repressor, and wherein the second recombinant nucleic acid is located in a non-essential region of the vaccinia virus genome or in an intergenic region of the vaccinia virus genome, and
wherein the conditional replication gene product is a gene product that is toxic to a mammalian host or cells, lowers the fitness of the recombinant vaccinia virus, or interferes with the replication of VACV, and
wherein expression of the conditional replication gene product is inducible or repressible by a tetracycline antibiotic.

21. A method of making a high titer virus preparation of a highly-attenuated vaccinia virus, comprising culturing the recombinant vaccinia virus of claim 1 in a medium comprising a tetracycline antibiotic, purifying the recombinant vaccinia virus in the absence of a tetracycline antibiotic to produce a recombinant vaccinia virus preparation, and titering the recombinant vaccinia virus preparation in the presence of a tetracycline antibiotic.

22. The method of claim 21, wherein the recombinant vaccinia virus further comprises an exogenous gene operably linked to an expression control sequence for the exogenous gene, wherein the product of the exogenous gene is a therapeutic protein.
